(19) United States
(12) Patent Application Publication
(10) Pub. No.: US 2024/0216628 A1
(43) Pub. Date: Jul. 4, 2024

(54) GAS INHALATION DELIVERY DEVICES AND RELATED METHODS

(71) Applicant: Duke University, Durham, NC (US)

(72) Inventors: Bastiaan Driehuys, Chapel Hill, NC (US); Alexander Church, Durham, NC (US)

(21) Appl. No.: 18/558,203

(22) PCT Filed: Apr. 21, 2022
(86) PCT No.: PCT/US2022/025668

§ 371 Date: Oct. 31, 2023

Related U.S. Application Data
(60) Provisional application No. 63/185,053, filed on May 6, 2021.

(51) Int. Cl. A61M 16/00; A61M 16/08; A61M 16/20

(52) U.S. Cl. CPC A61M 16/0078; A61M 16/0816; A61M 16/0833; A61M 16/201; A61M 2205/073; A61M 2205/075; A61M 2205/8218

(57) ABSTRACT

Devices that allow serially controlled delivery of room air for practice inhalation and breath-hold procedures and a different (image contrast) gas for inhalation for a medical procedure include an actuator that is controllably, pneumatically operated by a user via a length of conduit.

### RELATED APPLICATIONS

This application claims the benefit of and priority to U.S. Provisional Application Ser. No. 63/185,053, filed May 6, 2021, the contents of which are hereby incorporated by reference as if recited in full herein.

### GOVERNMENT GRANTS

The invention was made with government support under Grant Numbers R01HL126771 and R01HL105643 awarded by the National Institutes of Health/National Heart, Lung & Blood Institute. The United States government has certain rights in the invention.

### FIELD OF THE INVENTION

The invention relates to gas delivery devices that are particularly suitable for inhalation delivery of gases for use in imaging, such as for MRI evaluations.

### BACKGROUND

Hyperpolarized (which may also be referred to in an abbreviated form“HP”) ¹²⁹Xe MRI is emerging as a valuable means of imaging lung structure and function. See Kruger et al. Functional imaging of the lungs with gas agents. Journal of Magnetic Resonance Imaging. 2016; 43(2):295-315; Matin et al. Chronic obstructive pulmonary disease: lobar analysis with hyperpolarized ¹²⁹Xe MR imaging. Radiology. 2016; 282(3):857-868, the contents of which are hereby incorporated by reference as if recited in full herein. Hyperpolarized ¹²⁹Xe MRI is used for high-resolution, non-invasive 3D imaging of pulmonary function and to obtain dynamic and static spectral parameters for assessing lung function. See, e.g., U.S. Pat. No. 8,911,709 and U.S. Patent Application Publication Number 2020/0022616, the contents of which are also incorporated by reference as if recited in full herein.

It can be important to ensure that image or spectroscopy data are highly repeatable for clinical evaluations. Thus, ensuring that patients begin their inhalation of HP gas from a well-defined lung volume, quickly and comfortably within the confines of the MRI scanner can facilitate such an outcome.

Conventionally, a single-use dose delivery bag with a pinch valve as shown in FIGS. 1A and 1B has been used for providing HP gas to a patient for inhalation during an MRI imaging session. The HP gas in the dose delivery bag can comprise about a liter of about 1% enriched ¹²⁹Xe, about 10% nitrogen and about 89% helium as a drug component of a drug-device combination product provided by Polarean, Inc., Durham, NC. Current methods of dose administration involve a technician such as a Clinical Research Coordinator (CRC) coaching the patient to take some preparatory room air breaths and then to exhale fully before inhaling the gas from the dose delivery bag. Once the preparatory room air breathing is completed, the technician then places the mouthpiece of the dose delivery bag in the patient's mouth, opens the pinch valve, and rapidly releases the contents of the bag, squeezing the bag to facilitate the entire content is inhaled while coaching the patient to breathe in rapidly, e.g., saying “breathe in . . . breathe in . . . breathe in” and after about 6-8 seconds of inhalation, say “hold it” which can be used to signal the MRI operator to initiate the scan. However, as shown in FIG. 1B, the delivery procedure is challenged by the confined space of the MRI machine, often requiring a coordinator to reach into the bore of the magnet to reach the patient's mouth and/or to release the pinch valve using a hand that is adjacent the mouth and nose of the patient. This can lead to significant delays and confusion, undesirably causing a patient to inhale some air before the dose, and/or change their lung inflation level.

There is a need for alternate devices that can deliver gas for inhalation for a medical procedure, such as an MRI imaging session.

### SUMMARY

Embodiments of the present invention provide a compact inhalation delivery device that can serially administer either room air (and vent to room) or image contrast gas in a controlled manner.

The dose delivery device includes a pneumatic actuator assembly. The actuator controls at least one valve that allows the patient to inhale room air during practice inhalation procedures and to inhale gas from a dose delivery bag for a “breath-hold” inhalation for an imaging session.

This device can allow a clinician or technical assistant such as an MRI technologist to remain outside of a magnet bore during imaging (potentially in a position to initiate an image scan and actuate the actuation assembly of the device) as well as allows the patient to keep the mouthpiece in their mouth or face mask on their face during practice inhalations of room air then actual dose administration.

Embodiments of the present invention are directed to an inhalation gas delivery device that includes: an inhalation delivery output member; a bag of gas for inhalation in fluid communication with the inhalation delivery output member; and an actuator assembly with an actuator member. The actuator member has a first position which allows room air to flow along a first (optionally enclosed) flow path to the inhalation gas output member and the actuator member has a second position which allows gas from the bag to flow along a second enclosed flow path to the inhalation gas output member.

The device can also include a conduit in fluid communication with the actuator assembly whereby a user pneumatically actuates the actuator member via the conduit to move from the first position to the second position.

The inhalation gas delivery device can be non-ferromagnetic and can be configured for use in an MRI scanner room.

The inhalation gas delivery device can vent expiration breaths of inhaled air and inhaled gas to atmosphere.

The actuator member can have a piston that travels a stroke distance in a range of about 5 mm and about 10 mm between the first and second positions.

The actuator assembly can have a housing that encloses at least part of a piston. The housing can have at least one air inlet (vent) downstream of the piston and the actuator assembly can close the at least one air inlet when the actuator member is in the second position.

The inhalation gas delivery device can further include a housing having an air vent closure member that is coupled to the actuator member. The air vent closure member can slide linearly in concert with the actuator member as the actuator member moves from the first position to the second position.

The gas in the bag can include hyperpolarized noble gas. The inhalation gas delivery device can be non-ferromagnetic.

The hyperpolarized noble gas can be ¹²⁹Xe.

The bag and the actuator assembly can reside a (short) distance in a range of about 0.1 inches (2.54 mm)-12 inches (30.4 cm) from the inhalation delivery output member.

The actuator assembly can have a housing with an internal chamber. The housing can have an outer wall with at least one air inlet (or vent). The actuator member can include a piston with a first segment having a first outer diameter and a second segment having a second outer diameter. The first segment can have a face with a seal or the face can cooperate with a component that has a seal that seals the gas from the bag from flowing through the piston when the piston is in the first position.

The first segment can have at least one gas intake inlet adjacent the face seal surface. The second segment can block the at least one air inlet when the actuator member is in the second position.

The second segment can have first and second seals axially spaced apart and extending about the outer diameter thereof,

At least one air inlet can reside between the first and second seals when the actuator member is in the second position.

The piston can have a flat circumferentially extending working surface that projects radially outward and extends between the first and second segments.

The inhalation gas delivery device can further include a length of conduit coupled to the housing and in fluid communication with a pressurized fluid source and in fluid communication with the working surface of the piston whereby pressurized fluid input into the housing from the conduit actuates the piston to move to the second position.

The actuator assembly can have a housing and the housing can have an outer wall with at least one air inlet. The piston resides in the housing and the piston comprises a proximal end that comprises a plurality of gas intake inlets that merge into an open channel downstream of the gas intake inlets and forming part of the second flow path.

The actuation assembly can have a housing with an internal chamber and a piston as the actuation member inside the internal chamber. The housing can have air inlets providing intake of room air for the first flow path. The piston can have gas intake inlets upstream of the air inlets and the piston can block the air inlets in the second position and the piston can seal the gas intake inlets when the piston is in the first position.

The inhalation delivery device can further include a pressurized fluid source comprising a syringe coupled to a length of conduit and to the actuator assembly or a compressible bulb coupled to a length of conduit and to the actuator assembly.

The inhalation delivery output member can have a single branch that serially defines part of the first flow path and part of the second flow path.

The inhalation delivery output member comprises a first branch that defines part of the first flow path and a separate second branch that defines part of the second flow path.

Other embodiments are directed to methods of providing inhalable gas for a medical procedure. The methods can include: providing a bag of inhalable gas; providing an inhalation delivery output member such as a face mask or mouthpiece; and connecting an actuator assembly to the bag and to the inhalation delivery output member. The actuator assembly and the bag reside within 0.1-12 inches (2.54 mm-30.48 cm) from a face of the patient. The method also includes; placing the inhalation delivery output member on a face over a nose and mouth or in a mouth of a patient; directing the patient to inhale practice room air breaths with the inhalation delivery output member on the face or in the mouth while an actuator member of the actuator assembly is in a first position providing open air inlets; and then actuating the actuating assembly to move the actuator member to a second position to close the air inlets and open gas intake inlets to provide a flow path that directs gas from the bag of inhalable gas to flow to the inhalation delivery output member for inhalation by the patient.

The medical procedure can be an MRI imaging session.

The actuator assembly can be manually actuated via a pressurized fluid source in communication with the actuator assembly via a conduit that can optionally have a length in a range of 1-12 feet or 1-10 feet.

The inhalable gas can include hyperpolarized ¹²⁹Xe gas.

Other devices and/or methods according to embodiments of the invention will be or become apparent to one with skill in the art upon review of the following drawings and detailed description. It is intended that all such additional devices and methods be included within this description, be within the scope of the present invention, and be protected by the accompanying claims.

### BRIEF DESCRIPTION OF THE DRAWINGS

Features of the present invention will be more readily understood from the following detailed description of exemplary embodiments thereof when read in conjunction with the accompanying drawings.

FIG. 1A is a digital photograph of a prior art dose delivery device.

FIG. 1B shows the dose delivery device of FIG. 1A in use with a patient in a bore of a magnet of an MRI scanner.

FIG. 2 is a top view of a dose delivery device according to embodiments of the present invention.

FIG. 3 is a top view of the dose delivery device shown in FIG. 2 illustrating alternate flow paths according to embodiments of the present invention.

FIG. 4 illustrates the dose delivery device in use with a patient and a technician according to embodiments of the present invention.

FIG. 5A is an enlarged side view of an example actuator assembly of the dose delivery device shown in FIG. 2 according to embodiments of the present invention.

FIG. 5B is an enlarged side view of the example actuator assembly of the dose delivery device shown in FIG. 5A with the air vents/inlets closed according to embodiments of the present invention.

FIG. 6 is a top view of another embodiment of a dose delivery system according to embodiments of the present invention.

FIG. 7A is a side perspective, section view of the actuator assembly shown in FIG. 5A, illustrating the air closure member and actuator member in respective first positions according to embodiments of the present invention.

FIG. 7B is a side view of the section view shown in FIG. 7A.

FIG. 7C is another side perspective, section view of the actuator assembly shown in FIG. 5A illustrating the air closure member and actuator member in a second operative position from the first operative position shown in FIG. 7A according to embodiments of the present invention.

FIG. 7D is a side view of the section view shown in FIG. 7C.

FIG. 8 is a schematic side view of another embodiment of a dose delivery system according to embodiments of the present invention.

FIG. 9A is a partial section view of a part of a dose delivery system with a piston in a first, home position according to embodiments of the present invention.

FIG. 9B is a partial section view of the part of the dose delivery system shown in FIG. 9A but with the piston in a second, extended position according to embodiments of the present invention.

FIG. 10 is an enlarged front, side perspective view of the piston shown in FIGS. 8, 9A and 9B according to embodiments of the present invention.

FIG. 11 is a rear view of the piston shown in FIG. 10.

FIG. 12 is an enlarged front, side perspective view of another embodiment of the piston shown in FIG. 10 according to embodiments of the present invention.

FIGS. 13A and 13B are side perspective view of a dose delivery system similar to the system shown in FIG. 8 but with the actuator assembly comprising a fluid port connected to a conduit and a pressurized fluid source according to embodiments of the present invention.

FIGS. 14A and 14B illustrate the conduit and user input providing part of the actuator control shown in FIGS. 13A and 13B, respectively.

FIG. 15 is an enlarged side perspective view of a part of the dose delivery system shown in FIGS. 13A and 13B.

FIG. 16 is a top view of another embodiment of a dose delivery system according to embodiments of the present invention.

FIG. 17 is a partially exploded side, perspective view of the dose delivery system shown in FIG. 16.

FIG. 18A is an enlarged top, perspective view of the actuator assembly and

part of the bag conduit shown in FIG. 16.

FIG. 18B is an enlarged side view of the actuator assembly and part of the bag conduit shown in FIG. 18A.

FIG. 19A illustrates the air flow path when the piston of the actuator assembly is in a first position.

FIG. 19B illustrates a gas flow path when the piston of the actuator assembly is in a second position.

FIG. 20 is an enlarged, side perspective view of the actuator assembly shown in FIG. 16.

FIG. 21 is a disassembled view of the actuator assembly shown in FIG. 20.

FIG. 22A is an enlarged cross-sectional view of the actuator assembly shown in FIG. 20 with the internal piston in a first position allowing room air to flow therethrough according to embodiments of the present invention.

FIG. 22B is an enlarged cross-sectional view of the actuator assembly shown in FIG. 22A with the internal piston in a second position blocking room air and allowing (hyperpolarized) gas to flow therethrough according to embodiments of the present invention.

FIG. 23 is an enlarged, side perspective cross-sectional view of the actuator assembly shown with the piston in the position shown in FIG. 22A.

FIG. 24A is a side, end perspective view of the example piston of the actuator assembly shown in FIG. 23.

FIG. 24B is an opposing end perspective view of the example piston shown in FIG. 24A.

FIGS. 25A-FIG. 25C are illustrations of a sequence of actions that can be used to position the dose delivery device, then deliver the gas from a bag while a patient is inside the scanner according to embodiments of the present invention.

FIG. 26 is a flow chart of example actions that can be used to deliver a dose of gas to a patient for a medical procedure according to embodiments of the present invention.

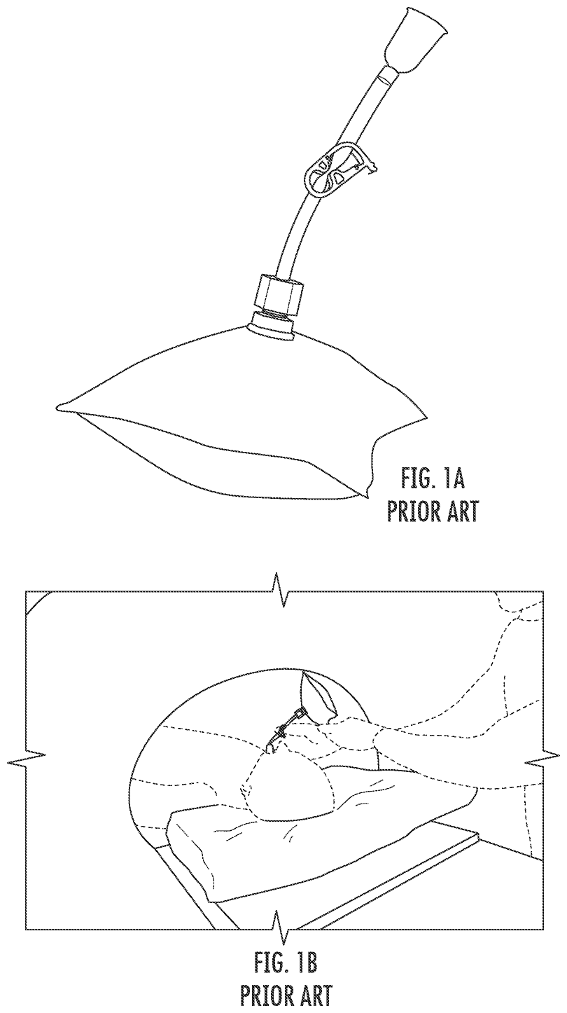

### DETAILED DESCRIPTION

While the invention may be made in modified and alternative forms, specific embodiments thereof are shown by way of example in the drawings and will be described in detail. It should be understood, however, that there is no intent to limit the invention to the particular forms disclosed, but on the contrary, the invention is to cover all modifications, equivalents, and alternatives falling within the spirit and scope of the invention. Like reference numbers signify like elements throughout the description of the figures.

In the figures, the thickness of certain lines, layers, components, elements or features may be exaggerated for clarity. Broken lines illustrate optional features or operations unless specified otherwise. The sequence of operations (or steps) is not limited to the order presented in the claims or figures unless specifically indicated otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

The term “MRI scanner” refers to a magnetic resonance imaging and/or NMR spectroscopy system. As is well known, the MRI scanners include a low field strength magnet (typically between about 0.1T to about 0.5T), a medium or a high-field strength super-conducting magnet, an RF pulse excitation system, and a gradient field system. MRI scanners are well known to those of skill in the art. Examples of commercially available clinical MRI scanners include, for example, those provided by General Electric Medical Systems, Siemens, Philips, Varian, Bruker, Marconi, Hitachi and Toshiba. The MRI systems can be any suitable magnetic field strength, such as, for example, about 1.5T or higher field systems of between about 2.0T-10.0T.

The term “high-field strength” refers to magnetic field strengths above 1.0T, typically above 1.5T, such as 2.0T or 3.0T. However, the present invention is not limited to these field strengths and may be suitable for use with lower or higher field strength magnets, such as, for example, 3.0T or even greater or even with other imaging modalities.

The term “hyperpolarized” refers to a noble gas such as ³He or ¹²⁹Xe that has increased polarization over natural or equilibrium levels. As is known by those of skill in the art, hyperpolarization can be induced by spin-exchange with an optically pumped alkali-metal vapor. See Albert et al., U.S. Pat. No. 5,545,396; and Cates et al, U.S. Pat. Nos. 5,642,625 and 5,809,801. These references are hereby incorporated by reference as if recited in full herein. One polarizer that is suitable for generating the hyperpolarized ¹²⁹Xe is the 9800, 9810 or 9820 polarizer models made by Polarean, Imaging, plc, Durham, NC. Thus, as used herein, the terms “hyperpolarize”, “polarize”, and the like mean to artificially enhance the polarization of certain noble gas nuclei over the natural or equilibrium levels.

Embodiments of the invention may be particularly suitable for use with human patients but may also be used with animals or other mammalian subjects.

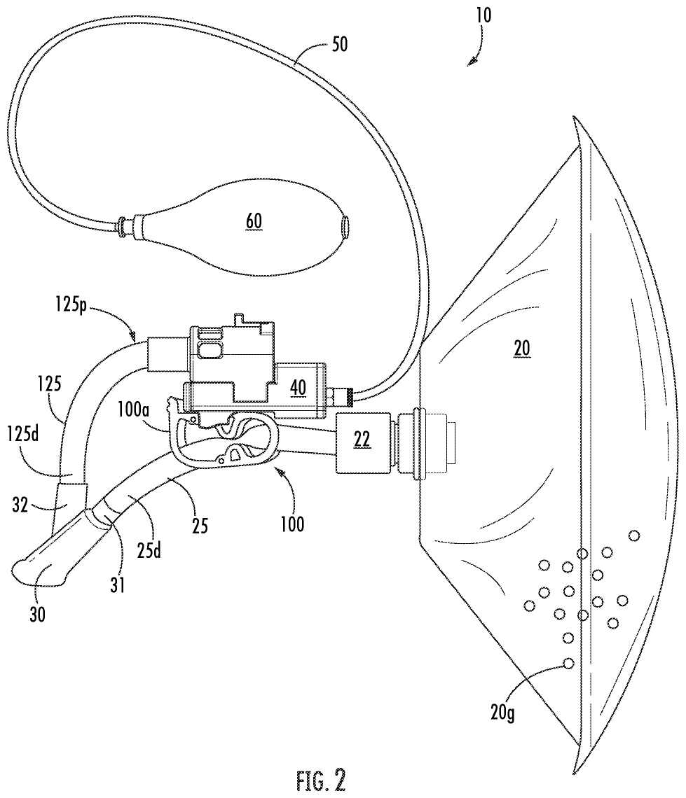

Turning now to FIGS. 2-4, 5A and 5B, an example dose delivery device 10 is shown. In the embodiment shown, the dose delivery device includes a bag 20 and a tube 25 sealably coupled to the bag 20 via a connector 22. The dose delivery device 10 also includes an inhalation delivery output member 30 sealably coupled to the tube 25 and an actuator assembly 40 in communication with a valve 100 that can controllably open and close the flow path 25f provided by the tube 25 between the gas 20g in the bag 20 and the inhalation delivery output member 30.

The bag 20 can be a medical grade Tedlar® film bag (from the polyvinyl fluoride family). See also, U.S. Pat. No. 6,128,918, the contents of which are hereby incorporated by reference as if recited in full herein.

The valve 100 can be a clamp or pinch valve that externally clamps/pinches the tube 25 to close the associated flow path 25f.

The inhalation delivery output member 30 can comprise a first branch 31 that couples to a distal end portion 25d of the tube 25 and a second branch 32 that couples to a distal end portion 125d of a secondary tube 125. The secondary tube 125 can define an airflow path 125f between room air and the inhalation delivery output member 30.

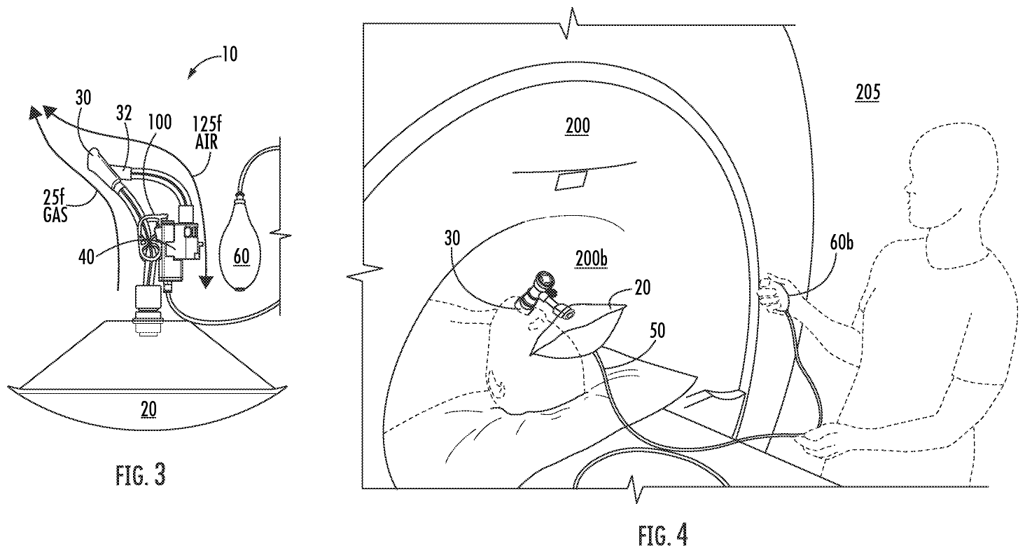

The conduit 50 can have a smaller inner and outer diameter than the tube 25 and/or tube 125 and can have a length that is longer than either of the tubes 25, 125. In some embodiments, the conduit 50 can have a (loose) length that is in a range of about 1 foot and about 12 feet, preferably 2-10 feet, more preferably 3-8 feet, such as about 3 feet, about 4 feet, about 5 feet and about 6 feet, about 7 feet and about 8 feet thereby allowing the user/technician to reside relatively close to the patient for “coaching” the breath hold procedure while the inhalation delivery output member 30 is in/on the patient with the bag 20 adjacent thereto and in operative position. The loose length of conduit 50 can allow the technician to reside a distance away from/outside the bore 200b of the magnet 200 and inside the scanner room 205 (FIG. 4) of an MRI suite.

The delivery device 10 can be configured to allow a single technologist to perform the procedure with a respective patient, including both actuating the actuator assembly 40 to deliver the dose of gas from the bag 20 for breath hold and initiating a scan during an MRI imaging session.

The conduit 50 can be flexible and extend down from a face of a user as it exits the magnet bore 200b. The bag 20 and actuator assembly 40 can reside within 0.1-12 inches of a face of a patient and/or from the inhalation delivery output member 30, e.g., close to the face of a patient, and adjacent to or partially or totally within the bore 200b as shown in FIG. 4.

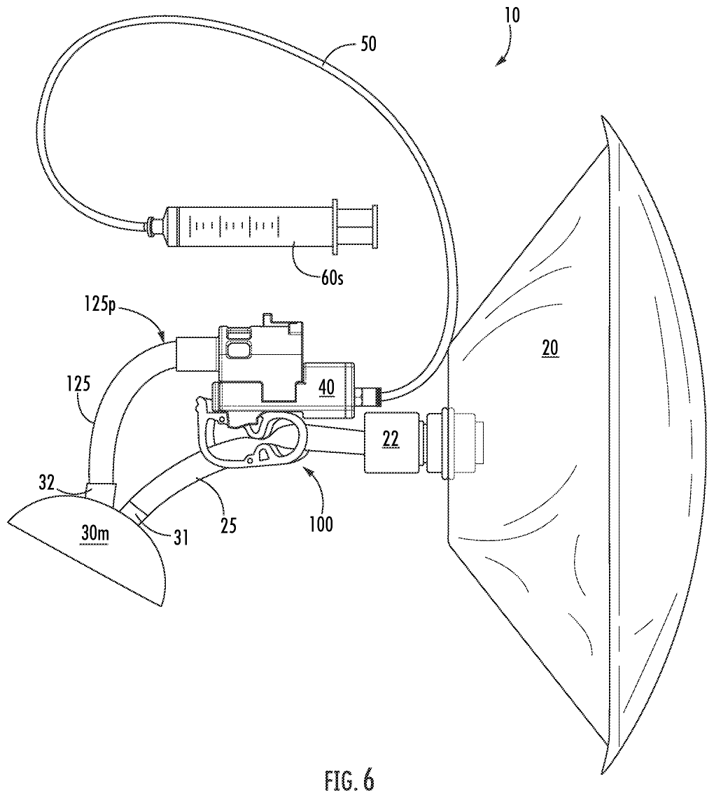

The conduit 50 can be in fluid communication with a pressurized gas source 60 such as a bulb 60b (FIGS. 2-4) or syringe 60s (FIG. 6).

The actuator assembly 40 can have a first connector 50c that couples to a conduit 50 that connects to a pressurized gas source 60 that provides pneumatic input to the actuator assembly 40. The actuator assembly 40 can have a second connector 125c that connects to a proximal end portion 125p of the secondary tube 125 providing the air input/output paths. The second connector 125c can reside in-line with and proximate to at least one air inlet 43 (interchangeably referred to as “air vent” herein), shown as a plurality of adjacent air inlets 43.

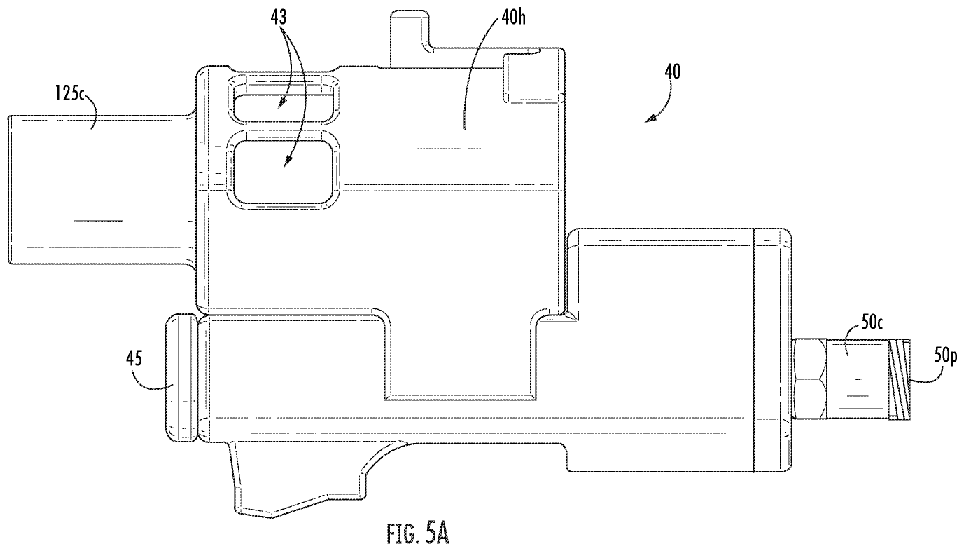

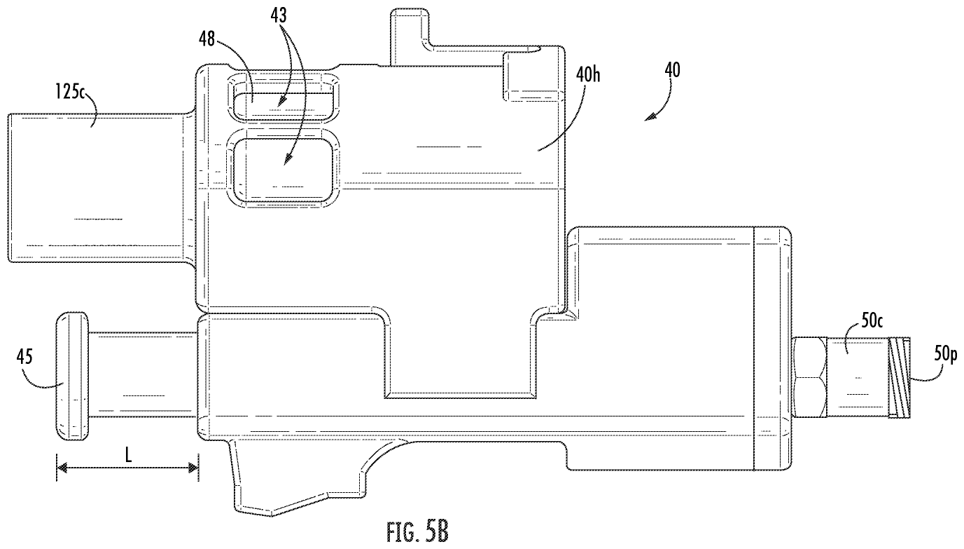

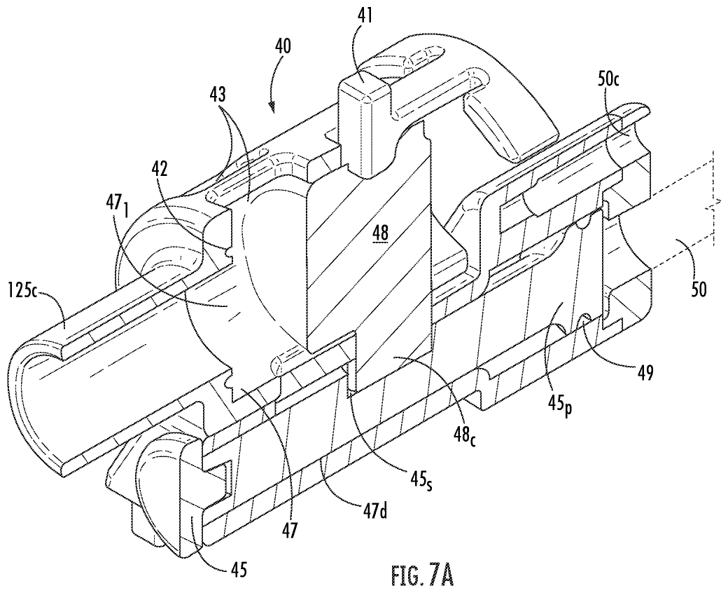

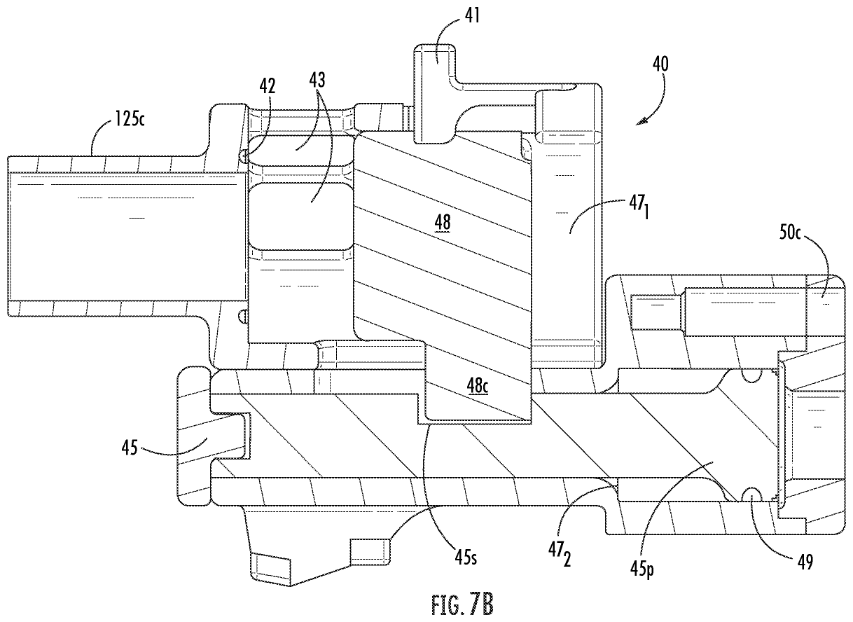

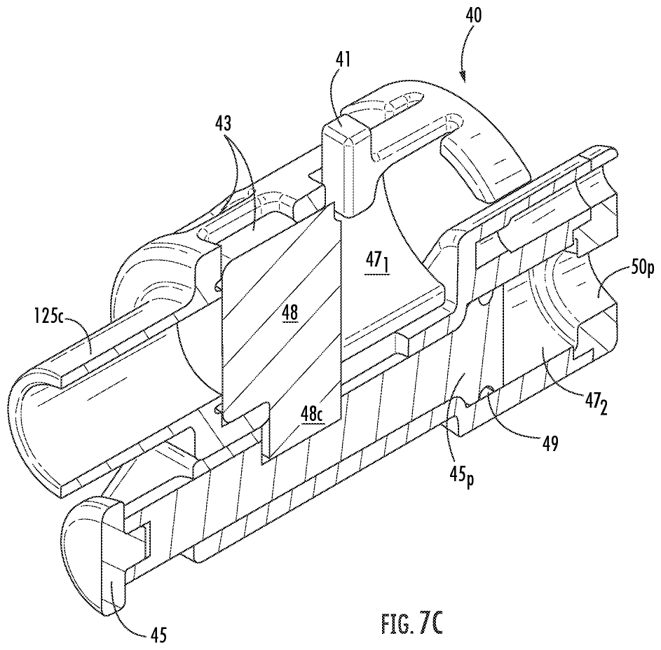

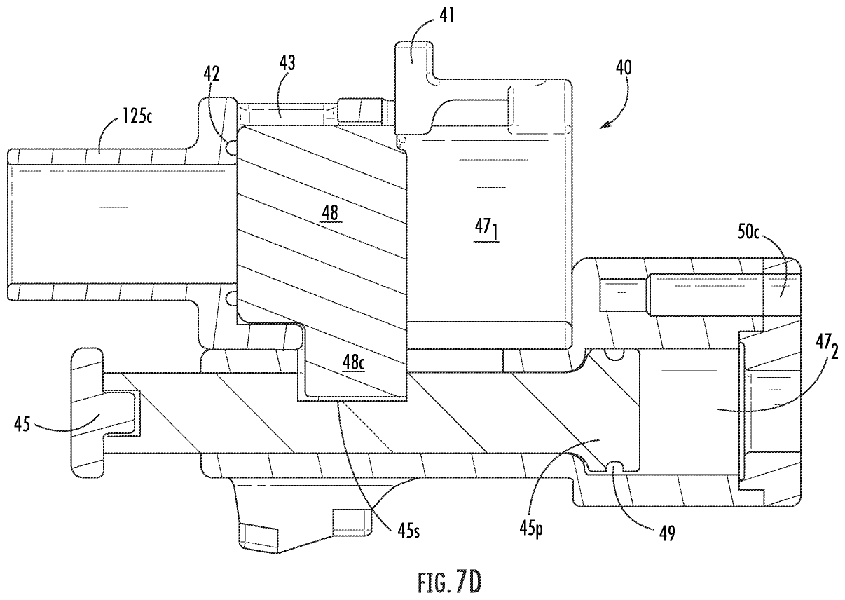

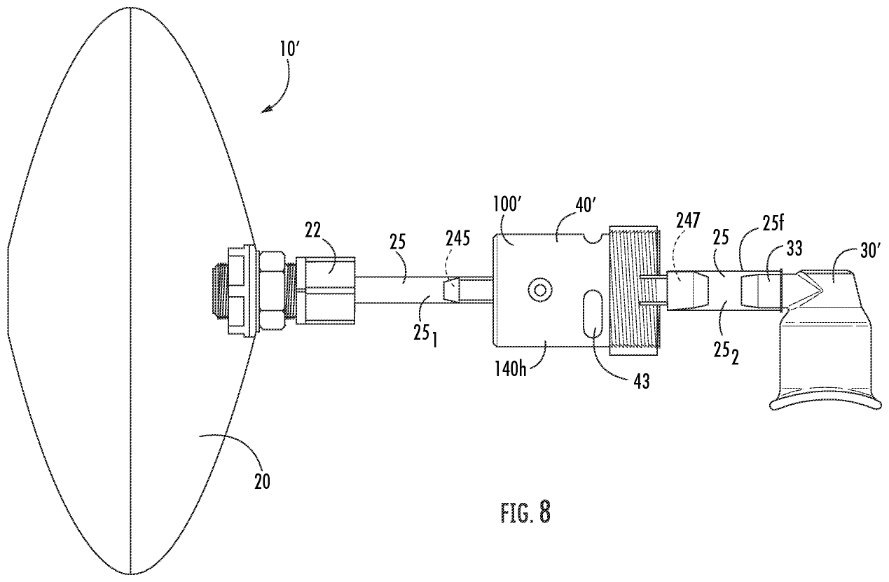

Referring to FIGS. 5A and 5B, the actuator assembly 40 can comprise a housing 40h and an actuator member 45 that has a linear stroke distance L. The actuator member 45 can be configured as and/or comprise a piston 45p (FIG. 7A) in fluid communication with the conduit 50. The stroke distance L can be relatively small such as a range of about 4-30 mm, more typically a range of about 5-10 mm such as about 8.5 mm, in some embodiments, for example. The actuator member 45 can have a first (home) position which is a retracted position whereby the valve 100 is pinched to close the flow path 25p. The actuator member 45 can have a second extended position (FIG. 5B) whereby the valve 100 is un-pinched and opens the flow path 25. Thus, when extended, the actuator member 45 pushes against an arm 100a of the valve 100 to decompress the tube 25 sufficiently to open the flow path 25f.

In some embodiments, the actuator assembly 40 can be configured, simultaneous with the extension of the actuator member 45, to direct an air (inlet) closure member 48 to move to close the air inlets 43 thereby preventing the patient from inhaling air through the inhalation delivery output member 30. The air closure member 48 can be coupled to the actuator member 45 and may slidably move axially parallel to and in concert with the actuator member 45.

Referring to FIGS. 7A, 7B, 7C and 7D, the actuator assembly 40 has first and second internal chambers 47₁, 47₂, one, above the other, the first chamber 47₁ enclosing the air closure member 48 and the second chamber 47₂ enclosing the piston 45p. The piston 45p can be sealably coupled to the second chamber 47₂ via a seal 49, such an O-ring, on the piston 45p adjacent the connector 50c for the conduit 50.

The actuator assembly 40 can also include a seal 42 configured as a face seal for closing/sealing the air closure member 48 against the distal end 47d of the chamber 47₁.

Still referring to FIGS. 7A-7D, the actuator assembly 40 can include a locking pawl 41 that locks the air closure member 48 in the closed position (FIGS. 7C, 7D) once the actuator assembly 40 is actuated to open the valve 100. The locking pawl 41 can be configured to block a proximal end of the air closure member 48 to keep the air closure member 48 from retracting.

The air closure member 48 can include a coupler segment 48c that is coupled to the actuator member 45. The coupler segment 48c can slidably reside inside the second chamber 47₂, inside a slot 45s formed in the actuator member 45.

Referring to FIG. 3, the example alternate flow paths 25f, 125f are shown with the appended arrows. The preparatory air flow breaths are allowed via tube 125 for air flow path 125 with air in-taken into the flow path 125f via the air inlets 43 (FIG. 5A) while the valve 100 closes the flow path 25f. When the actuator assembly 40 opens the valve 100, it closes the air inlets 43 so that the patient inhales the gas from the bag 20 via flow path 25f. Thus, the actuation assembly 40 of the inhalation delivery device 10 is actuated to close the air inlets (vents) 43 when positive pressure is applied.

In some embodiments, pressure can be (manually) applied externally to compress the bulb 60b (FIG. 2) or move a plunger of a syringe 60s (FIG. 6) to provide positive pressure to keep air inlets 43 (FIG. 5A) open to room air while the actuator member 45 is in the home or retracted (first) position.

Releasing or decreasing external pressure on the bulb 60b or retracting the plunger in the syringe 60s can reduce air pressure applied to the actuator assembly 40 which can cause the actuator assembly 40 to move an air inlet closure member 48 to close the air inlets 43 and concurrently extend the actuator member 45 (FIG. 5B).

The actuator assembly 40 can be configured to operate in the reverse whereby positive air pressure can be increased to move the actuator member 45 to the second position and close the air inlets 43.

In some embodiments, the device 10 can be configured to have the technician retract the syringe 60s or squeeze/compress the bulb 60b in order to close the flow path 25f at the bag outlet to the flow path 25f and open the air vents 43 once the scan is complete. This can allow the patient to exhale through the output member 30 of an intact device 10 once again after the scan. Also, or alternatively, negative pressure may be controllably applied to the actuator assembly 10 and used for redundancy at defined times/actions before or after the positive pressure is applied.

Typically, the technician squeezes the bulb 60b or extends a plunger the syringe 60s to administer the dose or actuate the device to close the air inlets 43 and open the flow path 25f from the bag 20 to the output member 30 when the patient is ready for the breath-hold inhalation of the gas after practice inhalations with room air, all the while the output member 30 is in position on/in the patient.

The inhalation delivery output member 30 can alternatively be configured as a face mask 30m (FIG. 6), which may be particularly suitable for pediatric use.

The actuator assembly 40 can be configured to provide linear motion of the actuator member 45 and the air closure member 48 in response to pneumatic input to open the valve 100 and concurrently seal the air inlets 43. The actuator assembly 40 is configured to allow the patient to inhale room air during practice inhalation procedures using the inhalation delivery output member 30, then close the air flow path off and open the gas path from the bag 20 to inhale the gas.

The dose delivery device 10 can be configured to be a compact, MRI-safe device that can administer hyperpolarized gas in a controlled manner. As shown in FIG. 3, this device 10 allows for the technologist to remain outside of the bore 200b of a super conducting magnet 200 during imaging. The dose delivery device 10 is configured so that the inhalation delivery output member 30 can remain in position on/in the patient both when practicing inhalation procedures using room air and during dose administration thereby facilitating imaging repeatability and proper dose inhalation.

Turning now to FIGS. 8-13A, 13B, another embodiment of a dose delivery device 10′ is shown. As shown, the dose delivery device 10′ comprises an actuator assembly 40′ that is configured to combine functions of the actuator assembly 40 and valve 100 described above into a single multi-functional piston 140 inside a piston housing 140h.

The piston housing 140h comprises a piston chamber 240 that encloses the piston 140. The piston housing 140h comprises at least one (room) air inlet 43, shown as a plurality of circumferentially spaced apart and circumferentially extending inlets 43 that are closer to the distal end portion 140d of the housing 140h than the proximal end portion 140p of the housing 140h.

The piston housing 140h has an attachment segment or member (connector) 245 that is directly or indirectly coupled to the connector 22 of the bag 20. The attachment segment 245 may be located on a proximal end portion 140p of the piston housing 140h.

The piston housing 140h can have a distal end portion 140d with a connector 247 that is directly or indirectly coupled to the inhalation delivery output member 30′. The output member 30′ can be formed to be integral with the connector 247 or even the piston housing 140h. As shown, the output member 30′ is a mouthpiece 30m.

The inhalation delivery output member 30′ can have a single branch 33 that is connected to the connector 247 of the piston housing.

Referring to FIG. 8, the dose delivery device 10′ can have a first tube segment 25₁ that is coupled to the proximal end portion 140p of the piston housing 140h. The first tube segment 25₁ can be in-line with the connector 245 and/or axially extending centerline of the piston 140. The first tube segment 25₁ can receive the connector 245 and couple to the connector 22 of the bag 20.

The dose delivery device 10′ can have a second tube segment 25₂ that is coupled to the distal end portion 140d of the piston housing 140h. The second tube segment 25₂ can be spaced apart from but in-line with the first tube segment 25₁. The second tube segment 25₂ receives the connector 247 and the single branch 33 of the inhalation delivery output member 30′.

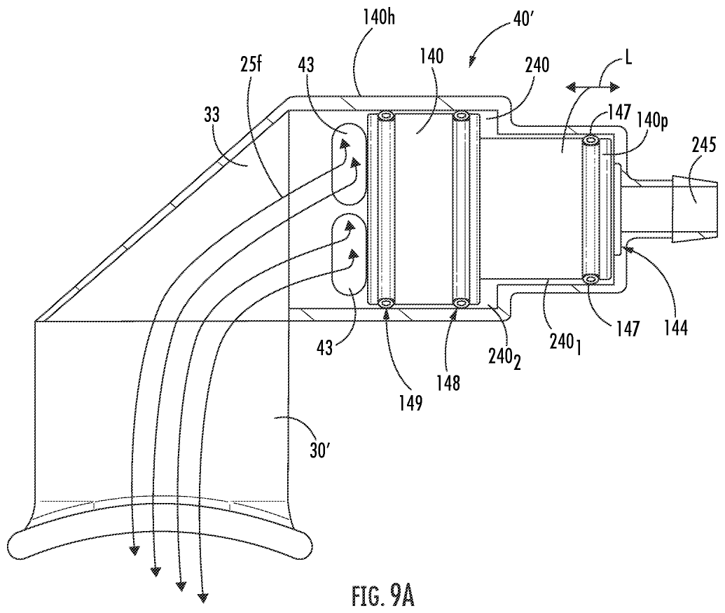

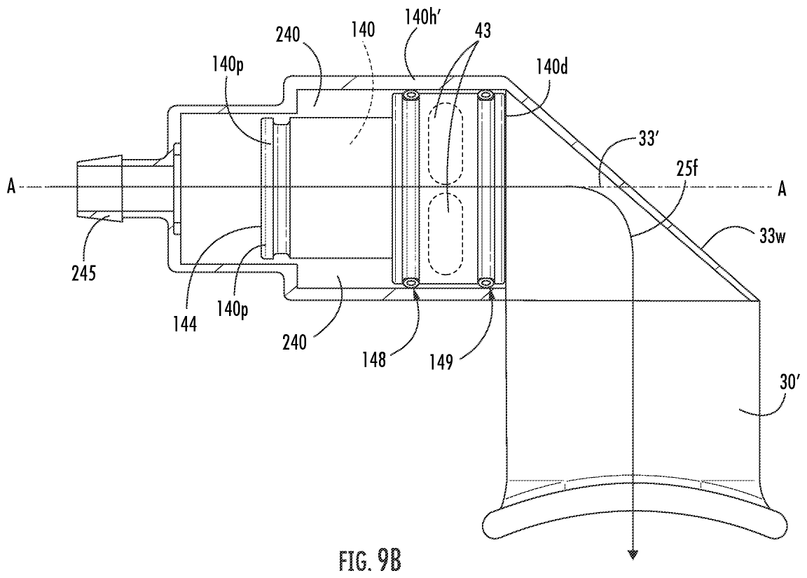

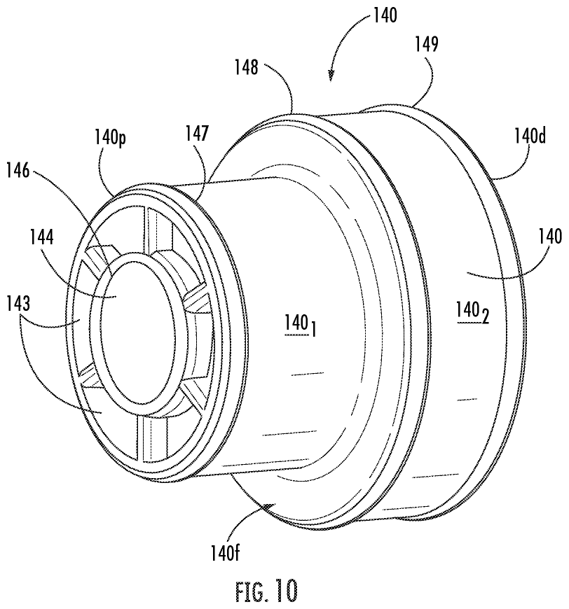

FIG. 9A illustrates the piston 140 in a home position, sealed against the proximal end portion 140p of the piston housing 140h, exposing the air inlets 43 allowing (room) air flow 25f between the room and the patient inhalation delivery member 30′. FIG. 9B illustrates the piston 140 in a second operative position, whereby the piston 140 sealably closes (blocks) the air inlets 43 and directs gas flow 25f from the bag 20 through gas inlets 143 in the piston 140 (FIG. 10) to the patient inhalation delivery member 30′.

FIGS. 9A and 9B also show the piston housing 140h and the patient inhalation delivery output member 30′ can be directly attached or integrated into a single monolithic body without requiring a segment of tubing to couple the two features. The branch segment 33′ can have an outer wall that is angled at an angle in a range of 30-60 degree from a centerline axis A-A of the piston 140, typically about 45 degrees.

The piston 140 can be configured to move axially a relatively short stroke distance between the first and second positions. The stroke distance L can be in a range of about 4-30 mm, more typically a range of about 5-10 mm, such as about 8.5 mm, in some embodiments. This stroke distance of the piston 140 controllably directs either air (FIG. 9A) or gas from the bag 20 (FIG. 9B) to the patient.

In the first (home or start) position (FIG. 9A), the piston 140 seals the gas from the bag 20 from entering the actuator assembly 40′ while the air inlet(s) 43 are open.

Referring to FIG. 10, a face seal surface 144 can be provided on a distal or proximal end of the piston 140. The face seal surface 144 can be a closed surface, forming a seal surface, optionally surrounded by a seal 146 such as an O-ring. The face seal surface 144 can be configured to keep the contents of the bag 20 closed off from flow path 25f prior to the actuator member being actuated, typically by a technician. FIG. 12 illustrates that no face seal 146 is required on the piston 140. The seal 146, whether an O-ring or other suitable seal configuration can be provided in the connector 245 or other mating component of the face seal surface 144 of the actuator assembly 40′ of the device 10′, rather than on the face 144 of the piston 140. An example of another configuration of a seal 146 is an extruded and cut seal profile, for example, optionally held by a groove.

Referring to FIGS. 10-12, the gas intake apertures 143 can be circumferentially spaced apart and positioned about the medially located seal surface 144. The gas intake apertures 143 are configured to allow gas from the bag 20 to flow through the piston 140 and into the inhalation delivery output member 30′ when the piston 140 is in the second (actuated) position (FIG. 9B).

Referring to FIGS. 9A, 9B, 10 and 12, that the piston 140 can include first and second concentric body segments 140₁, 140₂. The first segment 140₁ can reside closer to the proximal end of the housing 140h than the second segment 140₂. The first and second concentric body segments 140₁, 140₂ can reside in a chamber 240 in the piston housing 140h that has corresponding first and second concentric body segments 240₁, 240₂. The first segment 140₁ of the piston can provide the gas intake apertures 143 and seal surface 144 and can provide an outer diameter seal 147. The second segment 140₂ of the piston 140 can have a larger outer diameter than the first segment 140₁. The second segment 140₂ of the piston 140 can define a piston working face segment 140f and support first and second outer perimeter axially spaced apart seals 148, 149. The second segment 240₂ of the chamber 240 of the housing 140h can provide the air inlets 43. The second segment 140₂ of the piston 140 can block the air inlets 43 when the piston 140 is in the second position (FIG. 9B) with the air inlets between seals 148, 149.

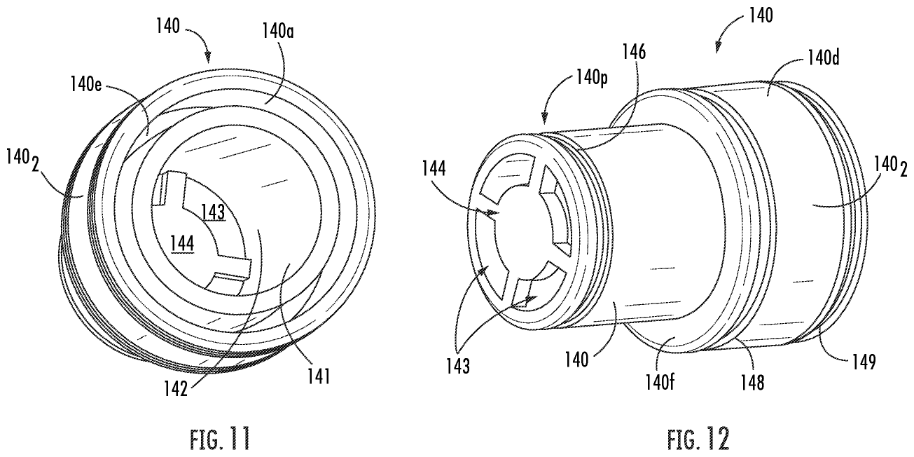

FIG. 11 illustrates a rear view of the piston 140 shown in FIG. 10. The piston housing 140h can include an internal (annular) cavity or chamber 140a extending rearward of the gas intake apertures 143 and/or about a length of the second segment 140₂ of the piston 140. The inner wall 141 of the annular chamber 140a can bound an axially extending open channel 142. The gas intake apertures 143 can merge into the open channel 142. A closed end 140e of the annular chamber 140a can reside radially outward from the open channel 142.

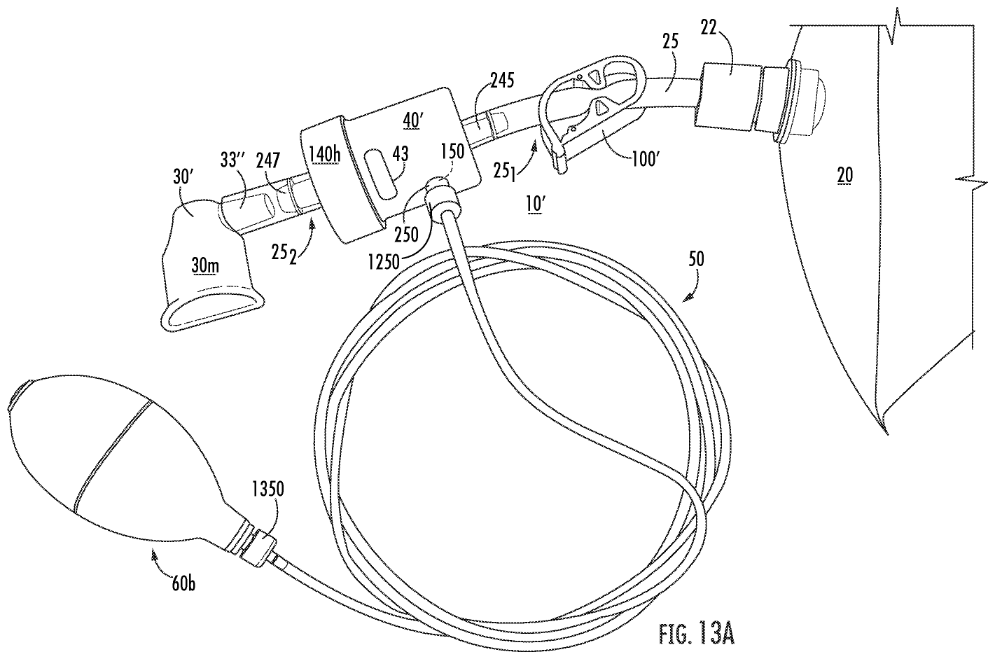

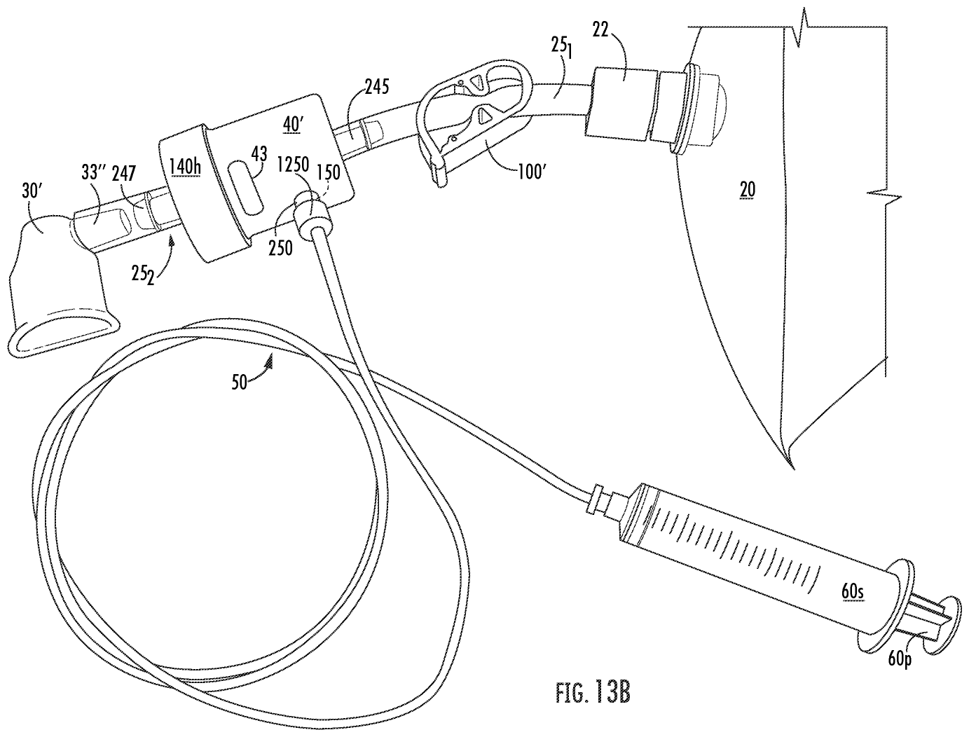

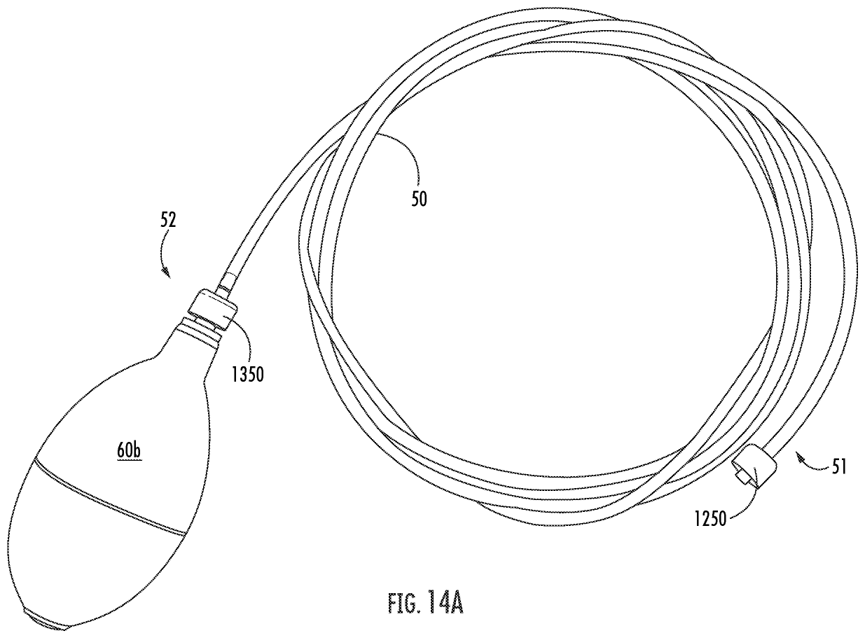

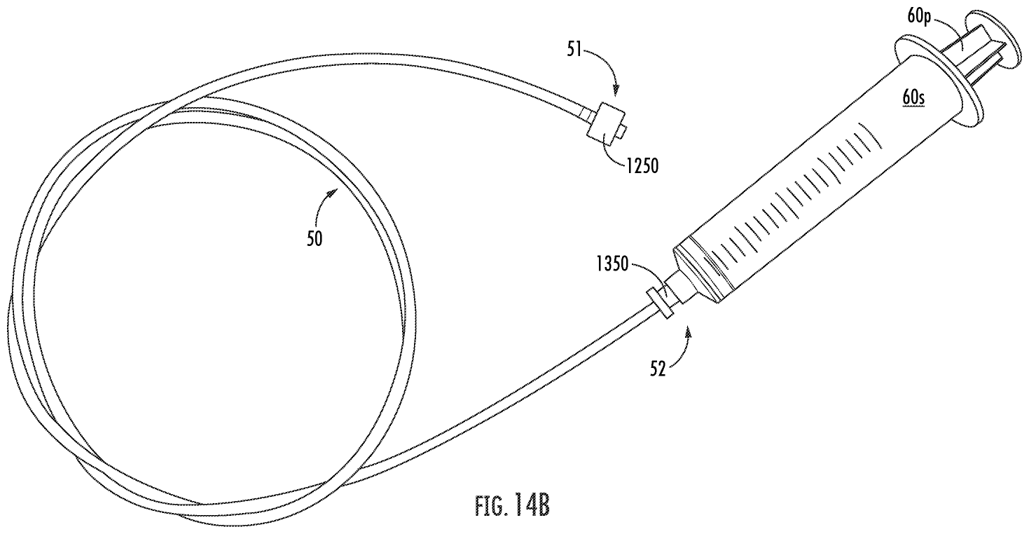

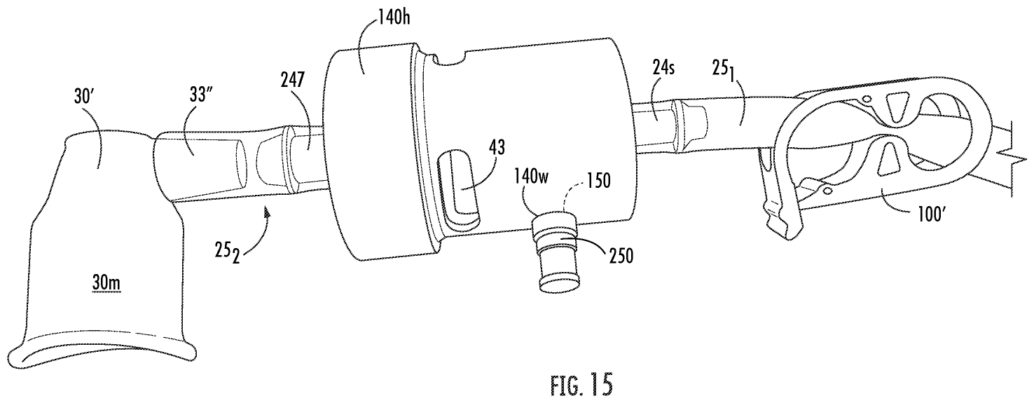

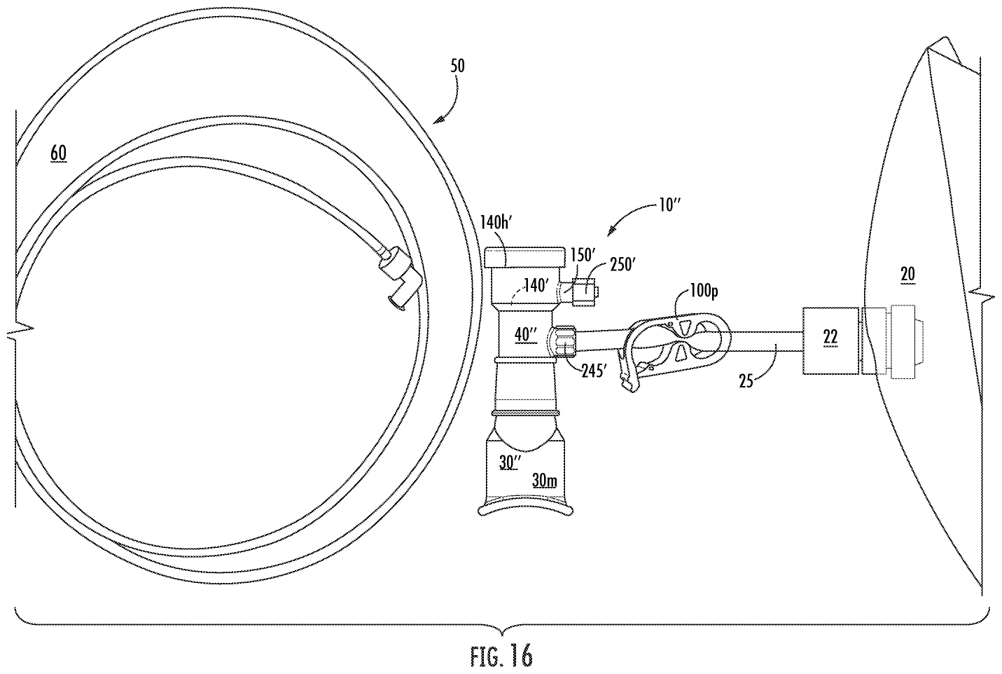

Referring to FIGS. 13A, 13B, 14A, 14B and 15, the inhalation (e.g., dose) delivery device 10′ can include an attachment segment/connector 250 coupled to the piston housing 140h and in fluid communication with a port 150 in an outer wall 140w of the piston housing 140h and to the piston working face 140f (FIG. 10) of the piston 140 inside the housing 140h. The attachment segment/connector 250 can be connected to a length of tubing 50 that is connected to a pressurized fluid source (typically a gas such as air) 60 such as a bulb 60b or syringe 60s as discussed above with respect to the embodiment of FIGS. 2 and 6. A first end portion 51 of the conduit 50 can include a first connector 1250 and a second end portion 52 of the conduit 50 can include a second connector 1350. The first connector 1250 can connect to the connector 250 provided by the piston housing 140h. The second connector 1350 can connect to the user input for the pressurized input for the actuator assembly 40, 40′. One or more of the connectors 250, 1250 and/or 1350 can be luer-lock connectors.

As shown, the conduit 50 can have a smaller inner diameter and outer diameter and a longer length than a sum of the first and/or second segments 25₁, 25₂ providing part of the inhalation flow path 25f between the bag 20 and the inhalation delivery output member 30, 30′.

A pinch valve 100p can be placed externally along the first segment 25₁ between the bag 20 and the piston housing 140h and used in combination with the actuator assembly 40′ for redundant control of the flow path 25f.

The branch 33′ of the inhalation delivery output member 30′ can extend into the second segment 25₂ of the flow path conduit 25 and can be in-line with the axially extending centerline of the piston 140.

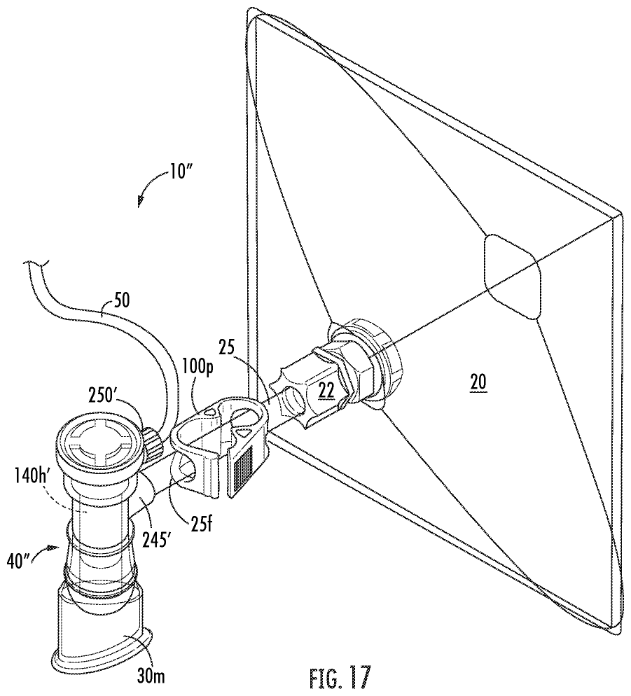

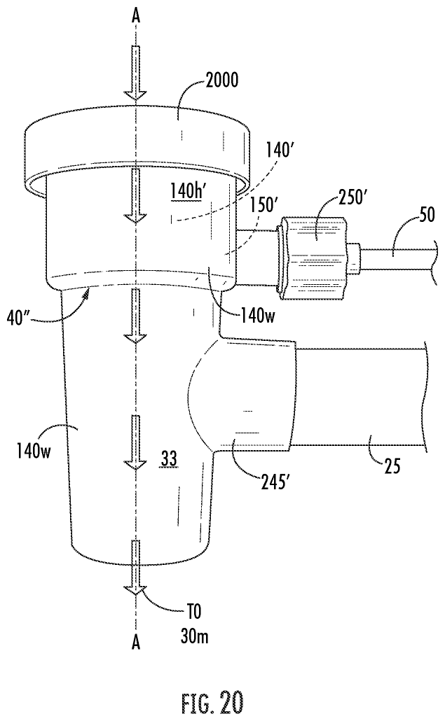

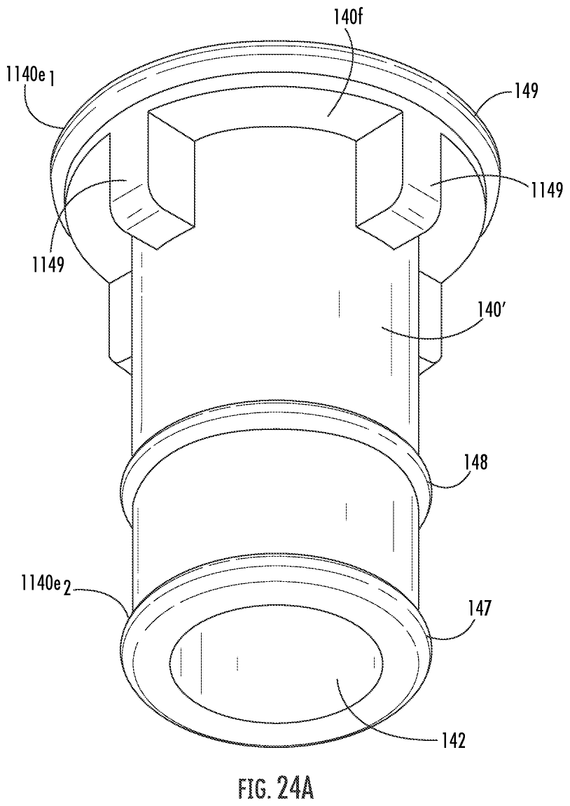

Referring now to FIGS. 16-24B, another embodiment of the dose delivery device 10″ with another example actuator assembly 40″ is shown. In this embodiment, the actuator assembly 40″ comprises a piston 140′ in a piston chamber 247′ in the piston housing 140h′. The piston chamber 247′ can have a straight linear configuration and the inhalation delivery device 30″ (shown as mouthpiece 30m) can be in-line with the piston chamber 247′ and the piston housing 140h′. As shown, the piston housing 140h′ defines a single branch 33 that connects to the inhalation delivery device 30′ and that can enclose the piston 140′. The piston 140′ can have an open channel 142 and the piston 140′ can travel between first and second operative positions in the piston housing 140h′.

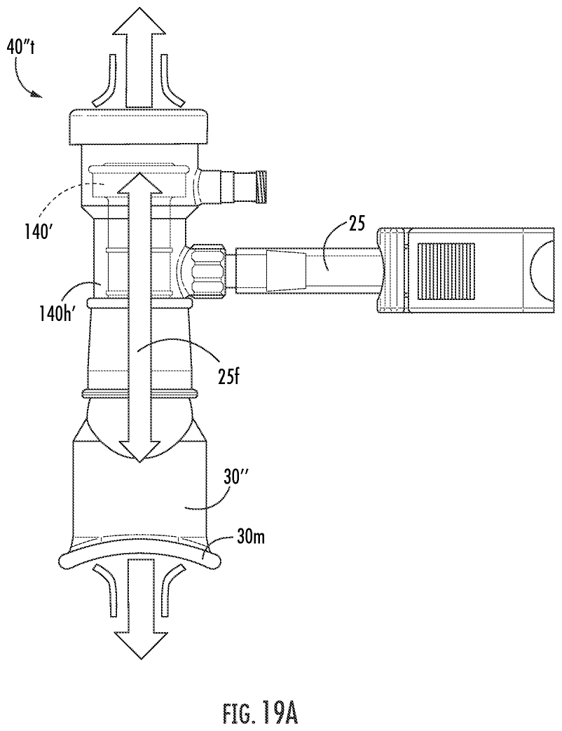

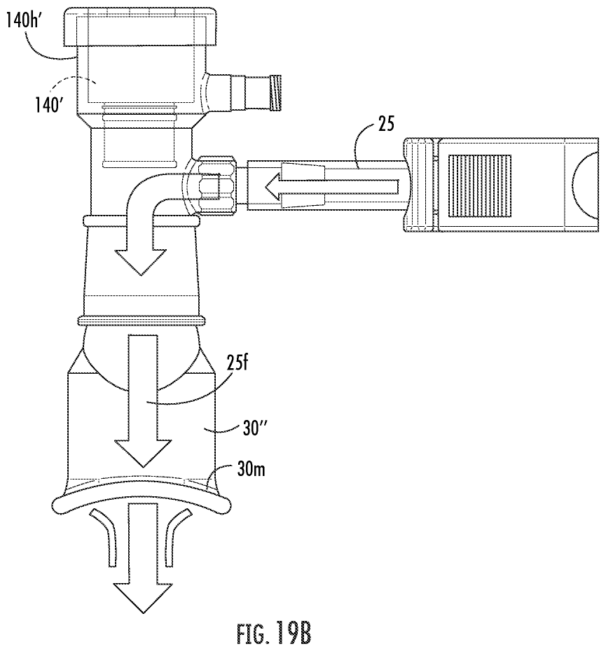

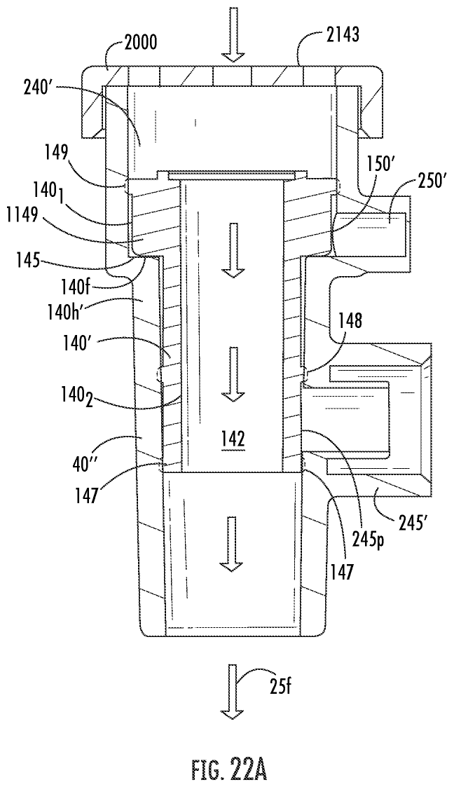

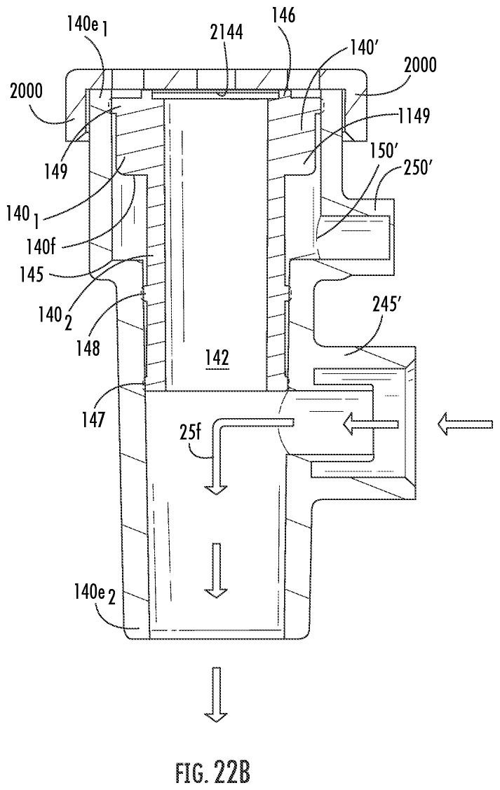

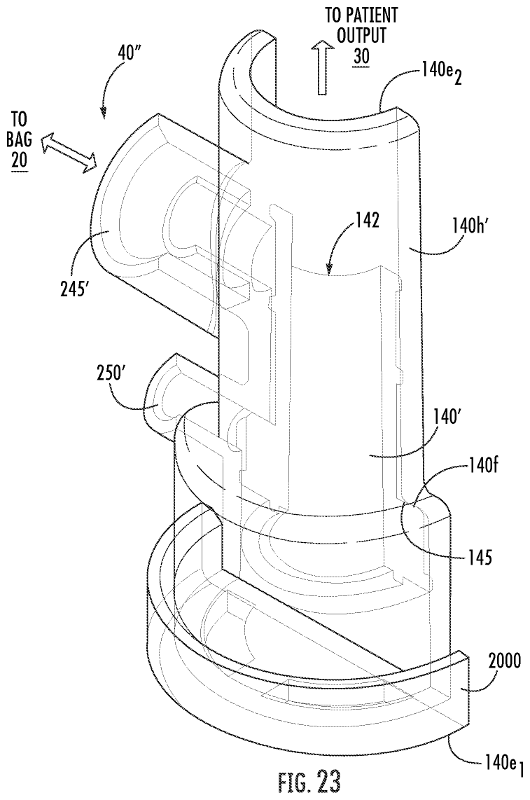

Referring to FIGS. 19A, 22A and 19B, 22B, the piston 140′ can move in the housing 140h′ between a first (home) position (FIG. 22A) to a second position (FIG. 22B). The piston 140′ is configured to cooperate with the housing 140h′ to allow (ambient) room air to flow through the piston 140′ to the inhalation gas output member 30′ (and flow the opposite direction to vent) when the piston 140′ is in the first position (FIGS. 19A, 22A). The piston 140′ is configured to cooperate with the housing 140h′ to block room air in the second position (FIGS. 19B, 22B) while also allowing gas from the bag 20 to flow in the flow path 25f provided by the housing 140h′ downstream of the piston 140′ to the inhalation gas output member 30′.

The housing 140h′ can have an outer wall 140w with first and second ports 150′, 245p extending therethrough. The first and second ports 150′, 245p can be provided as parallel projecting segments, connecting to conduits 50, 25, respectively. The piston housing 140h′ can have a first end portion 140e₁ that is axially spaced apart from a second end portion 140e₂. The second end portion 140e₂ faces the output device 30″, e.g., mouthpiece 30m. The first end portion 140e₁ intakes air and vents to atmosphere. The first end portion 140e₂ can include or be coupled to an end cap 2000.

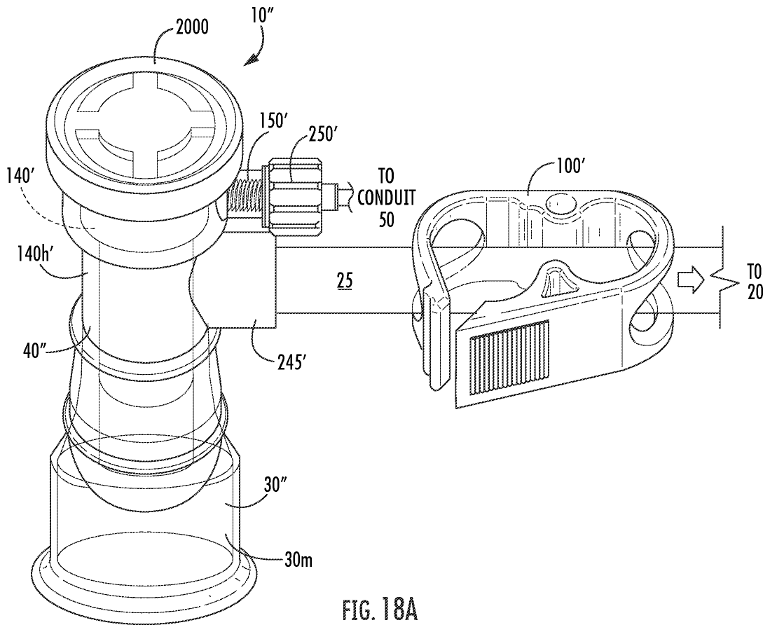

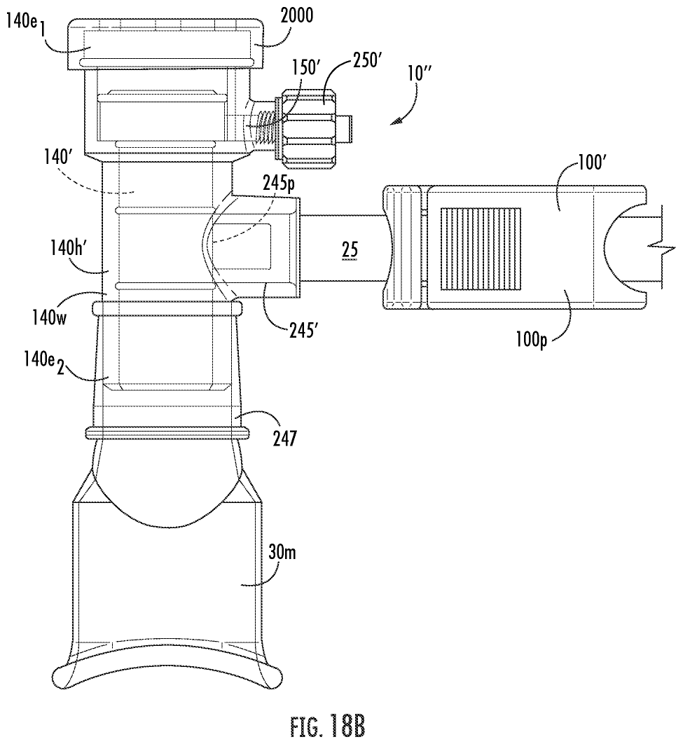

The second end portion 140e₂ can be coupled to the inhalation delivery output device 30″ via connector or adapter member 247 (FIG. 18B) that can attach the piston housing 140h and the mouthpiece 30m. The connector or adapter member 247 can be integral to the piston housing 140h, the mouthpiece 30m or provided as a separate connecting member.

The first port 150′ can be an actuator input port 150′ in fluid communication with the piston 140′. As shown, the first port 150′ is located further away from the gas inhalation output member 30″ than the conduit 25 attached to the bag 20. In operation, pressurized input forces the piston 140′ to move toward the first end portion 140e₁ of the housing 140h′ which closes air intake ports at the first end portion 140e1 of the housing 140h and concurrently opens the port 245′ to the conduit 25 attached to the bag 20 (FIG. 16).

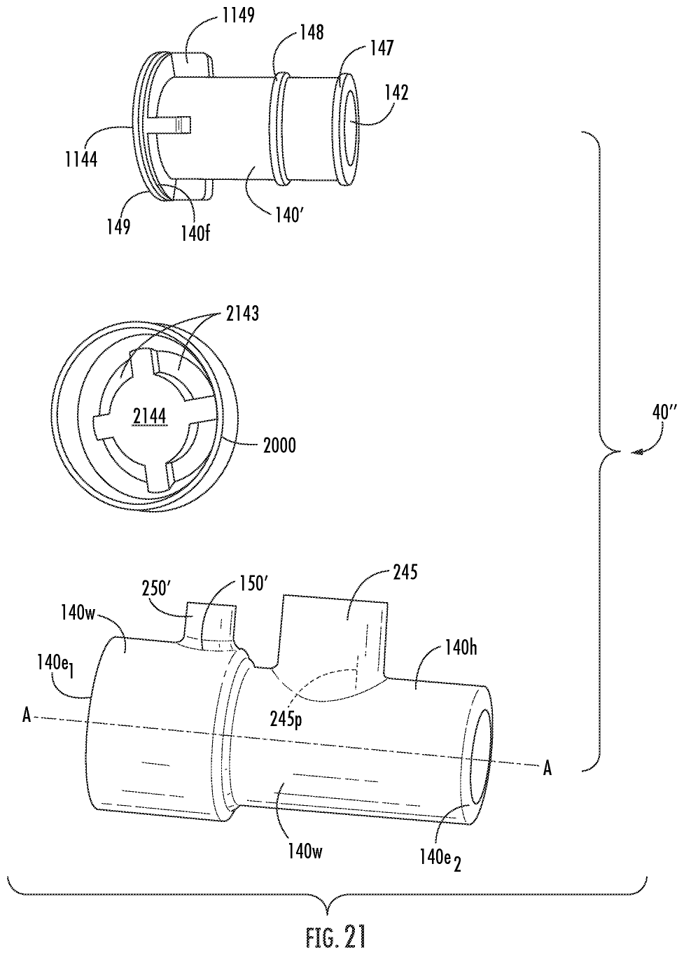

Referring to FIGS. 21, 22A, 22B, 23, 24A and 24B, the piston 140′ can have a plurality of circumferentially extending, axially spaced apart, seals 147, 148, 149. The seals 147, 148, 149 can be provided as elastomeric outer wall segments formed/injection molded/3-D printed/overmolded onto/into the wall 140w or body of the piston 140′. The seals 147, 148 149 may alternatively be provided as separate seal members such as O-rings or gaskets.

Referring again to FIG. 22A, the housing 140h′ can have a flat surface and/or ledge 145 that defines a seating surface for the piston 140′ when the piston 140′ is in the first position that blocks the port 245p.

The piston 140′ can have first and second longitudinally extending segments 140₁, 140₂, each with different outer diameters. The first segment 140₁ can have a greater outer diameter than the second segment 140₂. The flat surface 140f can define a step between the first and second segments 140₁, 140₂. The first segment 140₁ can reside further away from the inhalation delivery output member 30′ than the second segment 140₂ and can have a length that is less than 50% of the length of the second segment 140₂.

At least two of the seals, shown as first and second seals 147, 148, can be axially (longitudinally) spaced apart and extend circumferentially about the outer wall of the piston 140′. With the piston 140′ in the first position (FIG. 22A) one seal 148 resides on one side of the port 245p and the other seal 147 resides on the other side to thereby seal off the port 245p from the flow path 25f.

The piston 140′ can include at least one additional seal, shown as a third seal 149, that also extends circumferentially about the outer wall of the piston 140′, at a location closer to the first end 140ₑ₁ of the housing 140h. The seal 149 can reside on the first segment 140₁.

The piston 140′ can have a flat circumferentially extending working surface 140f that projects radially outward and cooperates with an internal flat surface 145 of the housing 140h when the piston 140′ is in the first position (FIG. 22A).

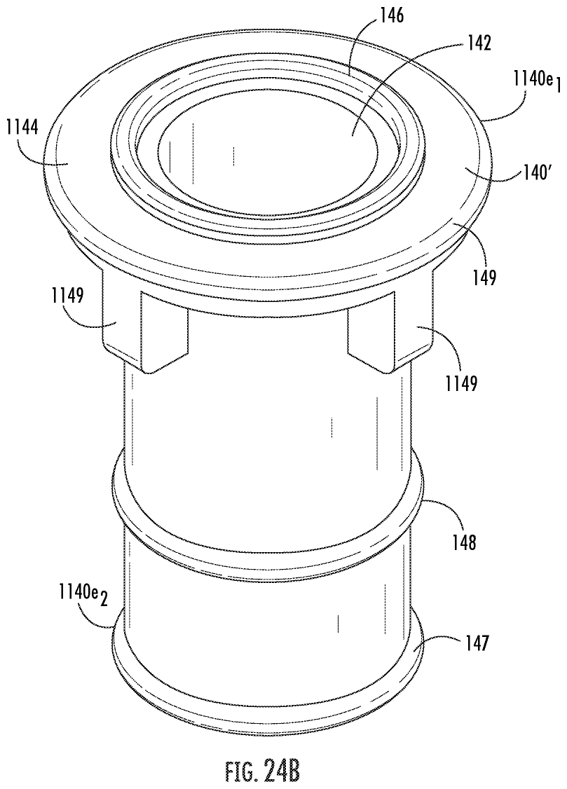

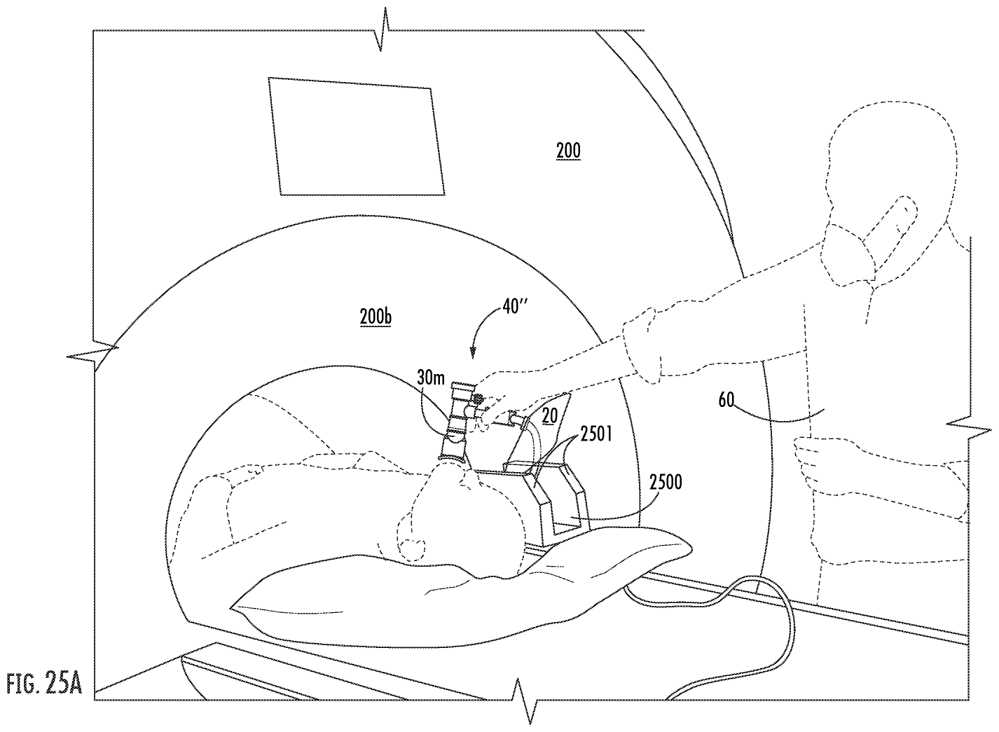

Referring to FIGS. 22A, 22B, 24A, 24B, the piston 140′ can have longitudinally extending legs 1149 that extend a sub-distance of the overall length of the piston 140′ to define a stop position against the ledge 145 for the piston 140′ in the piston chamber 240′. The legs 1149 can also provide structural rigidity to the piston 140′.

The top of the piston 140′ can also include an annular surface 1144 that resides in front of the legs 1149 and circumferentially extends about the opening for the channel 142.

Still referring to FIGS. 24A, 24B, the piston 140′ can have an open medial through channel 142. The first end 1140e₁ of the piston 140 can have a surface with a seal 146 that surrounds an outer diameter of the open channel 142 and projects outward from a seal face 1144. The first end 1140e₁ can also provide the seal 149. The seal 149 can extend circumferentially about the outer wall of the piston 140′. The seal 149 can reside in front of the legs 1149. The second end 1140e₂ can provide the circumferentially extending seal 147.

The seal 146 can seal against a cap 2000 providing a seal surface 2144 that faces the seal 146 and abuts the seal 146 when the piston 140′ is in the second position (FIGS. 19B, 22B). The cap 2000 also comprises at least one air intake/outlet aperture 2143 that allows air to enter and exit from the channel 142 of the piston 140′ when the piston 140′ is in the first position (FIGS. 19A, 22A). The gas from the bag 20 can flow through the piston housing 140h′ in the flow path 25f, downstream of the piston 140′, when the piston 140′ is in the second position (FIGS. 19B, 22B).

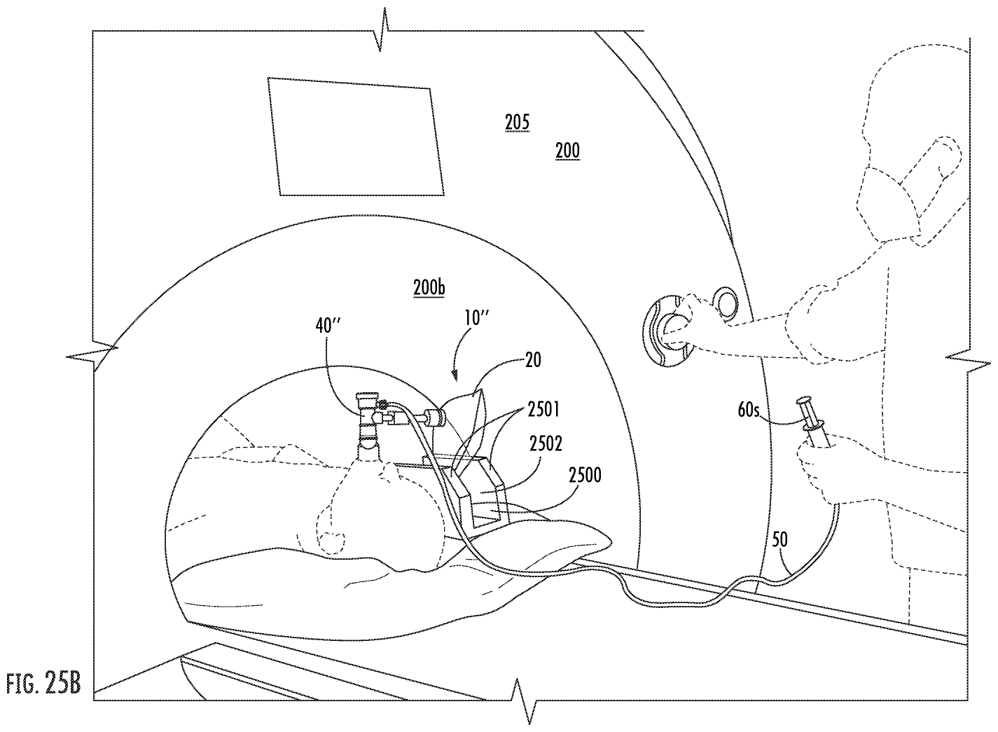

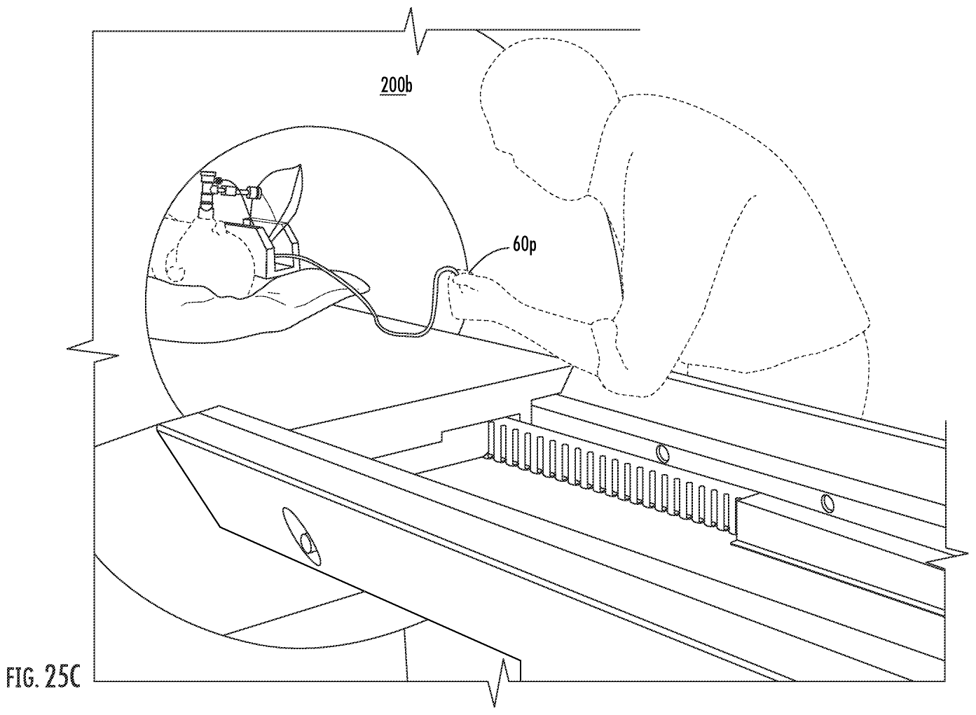

FIGS. 25A-25C illustrate a series of actions that can be used to dispense the gas from the bag 20 when the patient is in a bore 200b of a scanner 200 according to embodiments of the present invention.

FIGS. 25A-25C also illustrate the bag 20 can be at least partially supported by a bag holder 2500 to reduce any torque that may be applied to the mouthpiece 30m by the torque arm that may be generated by the conduit 25 and bag connector 22, for example. The bag holder 2500 is sized and configured to be able to move with the scanner bed without contacting the wall of the bore of the magnet.

The bag holder 2500 can be non-ferromagnetic and may have a pair of arms 2501 spaced apart to define a support space 2502 that receives a bottom portion of the bag 20. The bag holder 2500 can be configured to hold at least part of the bag 20 a distance above a table or bed of a scanner when the inhalation delivery output member is inserted into a patient to thereby reduce or remove any torque arm generated onto the inhalation delivery output member to reduce any retention force needed to be applied by the patient to retain the inhalation delivery output member in a mouth of the patient.

A clinician/technician can attach the length of tubing 50 and pressurized fluid source/syringe and then actuate the piston 140, 140′ once the patient is in position, e.g., within the MRI bore 200b and a patient has completed the preparatory breathing procedure using room air. The same clinician/technician can also initiate an MRI scan for obtaining MRI signal data of the patient using the inhaled gas such as image data and/or spectroscopic data of cardiac and/or lung function.

Where the device 10, 10′, 10″ is used to dispense hyperpolarized noble gas, the material(s)/components in communication with the gas should be medical grade and formulated to not negatively impact polarization decay and can also be non-ferromagnetic for MRI-compatibility.

The dose delivery device 10, 10′, 10″ can be a manually operated pneumatic control that is open loop and vents to atmosphere.

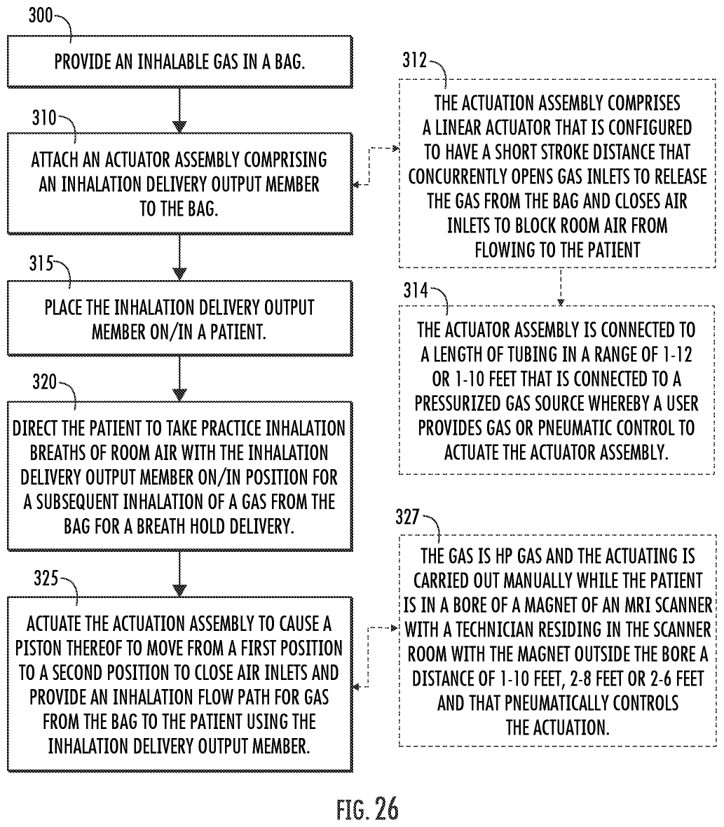

FIG. 26 is a flow chart of example actions that can be used to deliver an inhalation dose of a gas to a patient during an imaging procedure according to embodiments of the present invention. Inhalable gas is provided in a bag (block 300). An actuator assembly comprising an inhalation delivery output member is attached or attachable to the bag (block 310). The inhalation delivery output member is placed on/in a patient (block 315). The patient is directed to take practice inhalation breaths of room air with the inhalation delivery output member on/in position for a subsequent inhalation of a gas from the bag for a breath hold delivery (block 320). The actuation assembly is actuated to cause a piston thereof to move from a first position to a second position to close air inlets and provide an inhalation flow path for gas from the bag to the patient using the inhalation delivery output member (block 325).

The actuation assembly can comprise a linear actuator that is configured to have a short stroke distance that concurrently opens gas inlets to release the gas from the bag and closes air inlets to block room air from flowing to the patient (block 312).

The actuator assembly can be connected to a length of tubing typically in a range of 1-12 feet or 1-10 feet, such as in a range of 2-10 feet, 2-8 feet or 2-6 feet, for example, that is connected to a pressurized gas source whereby a user provides gas or pneumatic control to actuate the actuator assembly (block 314).

The gas can be HP gas and the actuating can be carried out manually while the patient is in a bore of a magnet of an MRI scanner with a technician residing in the scanner room with the magnet but outside the bore a distance of 1-10 feet (optionally 2-8 feet, such as 2-6 feet) to pneumatically control the actuation (block 327).

In some embodiments of the present invention have been illustrated herein by way of example. Many variations and modifications can be made to the embodiments without substantially departing from the principles of the present invention. All such variations and modifications are intended to be included herein within the scope of the present invention, as set forth in the following claims.

That which is claimed:

1. An inhalation gas delivery device, comprising:
an inhalation delivery output member;
a bag of gas in fluid configured for inhalation in fluid communication with the inhalation delivery output member; and
an actuator assembly comprising an actuator member coupled to the inhalation delivery output member, wherein the actuator member has a first position which allows room air to flow along a first flow path to the inhalation gas output member, and wherein the actuator member has a second position which allows gas from the bag to flow along a second enclosed flow path to the inhalation gas output member thereby blocking air from entering the second enclosed flow path.

2. The inhalation delivery device of claim 1, further comprising a conduit in fluid communication with the actuator assembly and a pressurized gas source whereby a user pneumatically actuates the actuator member, via the conduit, to move from the first position to the second position.

3. The inhalation gas delivery device of claim 1, wherein the inhalation gas delivery device is non-ferromagnetic and configured for use in an MRI scanner room.

4. The inhalation gas delivery device of claim 1, wherein the inhalation gas delivery device is configured to vent expiration breaths of inhaled air and inhaled gas to atmosphere.

5. The inhalation gas delivery device of claim 1, wherein the actuator member comprises a piston that travels a stroke distance in a range of 3 mm and 30 mm between the first and second positions.

6. The inhalation gas delivery device of claim 5, wherein the stroke distance is in a range of about 5 mm and about 10 mm.

7. The inhalation gas delivery device of claim 5, wherein the actuator assembly comprises a housing that encloses at least part of the piston, wherein the housing comprises at least one air inlet, and wherein the actuator assembly closes the at least one air inlet when the actuator member is in the second position.

8. The inhalation gas delivery device of claim 1, further an air vent closure member that is coupled to the actuator member, wherein the air vent closure member slides linearly in concert with the actuator member as the actuator member moves from the first position to the second position.

9. The inhalation gas delivery device of claim 1, wherein the gas in the bag comprises hyperpolarized noble gas, and wherein the inhalation gas delivery device is non-ferromagnetic.

10. The inhalation gas delivery device of claim 7, wherein the hyperpolarized noble gas is ¹²⁹Xe.

11. The inhalation gas delivery device of claim 1, wherein the bag and the actuator assembly reside a distance in a range of about 0.1 inches (2.54 mm)-12 inches (30.4 cm) from the inhalation delivery output member.

12. The inhalation gas delivery device of claim 1, wherein the actuator assembly comprises a housing with an internal chamber, wherein the housing comprises an outer wall with at least one fluid port, wherein the actuator member is a piston in the internal chamber of the housing, wherein the piston comprises a first segment having a first outer diameter and a second segment having a second outer diameter in the internal chamber of the housing, wherein the piston comprises an open channel extending therethrough, wherein the piston is configured to cooperate with the housing to (i) allow room air to flow through the piston to the inhalation gas output member when the piston is in the first position and (2) block room air while also allowing gas from the bag to flow to the inhalation gas output member when the piston is in the second position.

13. The inhalation gas delivery device of claim 12, wherein the second segment comprises first and second seals axially spaced apart and extending circumferentially thereabout, wherein the first segment comprises at least one seal extending circumferentially thereabout.

14. The inhalation gas delivery device of claim 12, further comprising a conduit attached to the bag and to the housing, wherein the at least one fluid port comprises an actuator input port in fluid communication with the piston.

15. The inhalation gas delivery device of claim 12, further comprising a length of conduit coupled to the housing and in fluid communication with a pressurized fluid source, wherein the length of conduit cooperates with the housing to direct pressurized fluid from the fluid source toward a working surface of the piston whereby pressurized fluid input into the housing from the conduit actuates the piston to move to the second position.

16. The inhalation delivery device of claim 1, wherein the actuator assembly comprises a housing, wherein the housing comprises an outer wall with first and second fluid ports, the first fluid port in fluid communication with the bag via a length of conduit and the second fluid port in fluid communication with a pressurized fluid source configured to actuate the actuator member to move to the second position.

17. The inhalation delivery device of claim 1, wherein the actuator assembly comprises a piston as the actuator member inside an internal chamber of a piston housing, wherein the piston seals air inlets of the inhalation delivery device in the second position and the piston seals an inhalation gas inlet of the inhalation delivery device when the piston is in the first position.

18. The inhalation delivery device of claim 1, further comprising a pressurized fluid source comprising a syringe coupled to a length of conduit and to the actuator assembly or a compressible bulb coupled to a length of conduit and to the actuator assembly.

19. The inhalation delivery device of claim 1, wherein the inhalation delivery output member comprises a single branch that serially defines part of the first flow path and part of the second flow path.

20. The inhalation delivery device of claim 1, wherein the inhalation delivery output member comprises a first branch that defines part of the first flow path and a separate second branch that defines part of the second flow path.

21. The inhalation delivery device of claim 1, wherein the actuator member comprises a piston with a plurality of longitudinally spaced apart seals that extend circumferentially about the piston, and wherein the actuator assembly is configured to allow air to flow into and/or out of the first enclosed flow path when the piston is in the first position and is configured to allow only gas from the bag to flow into the second enclosed flow path when the piston is in the second position.

22. The inhalation delivery device of claim 1, further comprising a bag holder configured to hold at least part of the bag a distance above a table or bed of a scanner when the inhalation delivery output member is inserted into a patient to thereby reduce or remove any torque arm generated onto the inhalation delivery output member to reduce any retention force needed to be applied by the patient to retain the inhalation delivery output member in a mouth of the patient.

23. A method of providing inhalable gas for a medical procedure, comprising:
providing a bag of inhalable gas;
providing an inhalation delivery output member such as a face mask or mouthpiece;
connecting an actuator assembly to the bag and to the inhalation delivery output member, wherein the actuator assembly and the bag reside within 0.1-12 inches (2.54 mm-30.48 cm) from a face of the patient;
placing the inhalation delivery output member on a face over a nose and mouth or in a mouth of a patient;
directing the patient to inhale practice room air breaths with the inhalation delivery output member on the face or in the mouth while an actuator member of the actuator assembly is in a first position providing open air inlets; and then
actuating the actuating assembly to move the actuator member to a second position to close the air inlets and open gas intake inlets to provide a flow path that directs gas from the bag of inhalable gas to flow to the inhalation delivery output member for inhalation by the patient.

24. The method of claim 23, wherein the medical procedure is an MRI imaging session, wherein the actuator assembly is manually actuated via a pressurized fluid source in communication with the actuator assembly via a conduit that has a length of 1-10 feet, and wherein the inhalable gas comprises hyperpolarized ¹²⁹Xe.

25. The method of claim 23, further comprising placing the bag of inhalable gas into a bag holder.

26. The method of claim 25, wherein the bag holder is non-ferromagnetic.
